(19) United States
(12) Patent Application Publication
(10) Pub. No.: US 2003/0083747 A1
(43) Pub. Date: May 1, 2003

(54) Bone implant and isertion tools

(75) Inventors: John Winterbottom, Jackson, NJ (US); Erik Martz, Howell, NJ (US); David R. Kaes, Toms River, NJ (US); Daniel Evan Rosenthal, Short Hills, NJ (US)

Correspondence Address: William Squire, Esq., c/o Carella, Byrne, Bain, Gilfillan, Cecchi,, Stewart & Olstein, 6 Becker Farm Road, Roseland, NJ, 07068, US

(73) Assignee: Osteotech, Inc.

(21) Appl. No.: 10/282,552

(22) Filed: Oct. 29, 2002

Related U.S. Application Data
(60) Provisional application No. 60/340,734, filed on Oct. 30, 2001.
(60) Provisional application No. 60/372,972, filed on Apr. 16, 2002.

(51) Int. Cl. A61F 2/44; A61F 2/46

(52) U.S. Cl. 623/17.11

(57) ABSTRACT

Spinal and other implants may be bone or synthetic material shaped as rings, C-shaped or rectangular and so on and also may have serrated wedge shaped top and bottom surfaces to match the disc space lordosis of adjacent ertebra and so on. The implants have one or more recesses aligned in an insertion direction at either or both outer peripheral sides of the implant. In a ring implant, e.g., formed from a transverse slice of the diaphysis of a long bone or otherwise, the recesses are aligned overlying the opposing sides of the ring parallel to the insertion direction, which sides are stronger than the more central region overlying and aligned with a central chamber in the implant. The alignment ith the opposing sides minimizes damage to the implant at the weaker more central region in response to implant insertion forces. One or two spaced recesses on opposite sides of an implant have surface(s) transverse to the insertion direction receive the tips of implant insertion gripping jaws for receiving an insertion load imparted by the tips. The recesses may have gripping surfaces extending in the insertion direction, which may be anterior/posterior or at an angle to the anterior/posterior direction for insertion laterally or anterior/laterally to the anterior/posterior direction. Such an implant may be non-bone and may be used to support or fuse bone not limited to the spine. Various embodiments of insertion tools and implants are disclosed.

### CROSS REFERENCE TO RELATED APPLICATIONS

This application claims priority on provisional applications serial No. 60/340,734 filed Oct. 30, 2001 and serial No. 60/372,972 filed Apr. 16, 2002.

This invention relates to bone implants, and particularly, but not limited to, spinal intervertebral fusion implants and insertion tools for insertion of implants into the intervertebral disc space, and more particularly, to anterior and posterior approach implants and tools.

Of interest are commonly owned copending application Ser. No. 09/705,377 entitled Spinal intervertebral Implant filed Nov. 3, 2000 in the name of Lawrence A. Shimp et al., Serial No. 60/246,297 entitled Spinal Intervertebral Implant Insertion Tool filed Nov. 7, 2000 in the name of Erik Martz et al. and Serial No. 60/264,601 entitled Implant Insertion Tool filed Jan. 26, 2001 in the name of John M. Winterboftom et al., and commonly owned U.S. Pat. No. 6,277,149, all incorporated by reference herein.

Surgical procedures for fusing adjacent vertebrae to treat various pathologies are well known. Implants for such procedures take a wide variety of shapes, forms and materials from bone to titanium, inert materials, rigid and elastic, circular cylindrical, wedge shapes, cages with or without openings to accept bone fusion promoting material. The surgical procedures may be posterior approach known as Posterior Lumbar Interbody Fusion (PLIF) or Anterior Lumbar Interbody Fusion (ALIF). The former procedure approaches the body from the rear and the latter approaches the body from the front by forming an opening in the abdomen to reach the spine. Also included is the TLIF (Transforaminal Lumbar Interbody Fusion), the anterior-lateral approach and the lateral approach. The latter two approaches approach the spine at a lateral angle (between 0° to 90°) or lateral (90°) to the anterior-posterior axis.

Because the anterior approach, in a spinal procedure, which is through the abdomen, needs to access the spine through a generally larger opening than the posterior approach, the tools for the anterior approach differ from those of the posterior approach. The implants also differ in configuration in the two approaches. The aforementioned applications and patent are concerned with the PLIF procedure.

The implants disclosed in the aforementioned copending applications is preferred for PLIF procedures. The implants, regardless the procedure, are dimensioned and shaped to provide a predetermined disc space between the adjacent vertebra to be fused.

Generally, bone growth promoting material is used in conjunction with the implant especially inert implants of metal, ceramic or other synthetic compositions. Often this growth promoting material is in the form of bone chips or bone fibers. These are not normally load bearing materials. Ground up mineralized cortical bone may be used for such chips, but has little bone growth factors. If bone marrow is mixed in the composition, then bone growth factors become present. Such material may be taken from the patient for use in the implant for that patient. The bone source for the chips and implant may be the iliac crest of the patient which is not desirable due to pain and long recovery periods.

C-shaped implants are described in the aforementioned copending applications and patent for use in the PLIF procedure.

Published PCT international applications WO 99/09914 and WO 00/24327 also disclose spinal C-shaped intervertebral implants for the PLIF procedure and is incorporated by reference herein.

U.S. Pat. No. 4,879,915 to Brantigan illustrates a spinal intervertebral implant. The implant is circular cylindrical and has a threaded bore and two opposing radial slots at one end for receiving an insertion tool threaded stud and prongs.

U.S. Pat. No 4,904, 261 to Dove et al. illustrates an inert C-shaped spinal fusion implant.

U.S. Pat. No. 5,192,327 to Brantigan discloses a prosthetic implant for vertebrae.

U.S. Pat. No. 5,443,514 discloses a method for fusing adjacent vertebrae using a spinal implant. The implant has through openings to provide for blood flow and bone growth from one side of the implant to the other side of the implant to adjacent vertebra. The implant is made of chopped fiber reinforced molded polymer, stainless steel or titanium. However, such materials do not permit direct bone in growth into the material and thus is a separate, discrete device which never forms a part of the bony structure of the spine except for the

bone in growth in the through openings.

U.S. Pat. No. 5,522,899 to Michelson discloses spinal implants which are substantially hollow rectangular configurations. In one embodiment, a series of implants are placed side by side in the intervertebral space to substantially fill the disc space. Autogenous bone material is packed within the hollow portion to promote bone growth. In other embodiments, a substantially rectangular implant member has a series of ridges on upper and lower surfaces. The material of the implants is not described.

U.S. Pat. No. 5,7669,897 to Harle discloses a wedge implant having a first component of a synthetic bone material such as a bioceramic material and a second component of a synthetic bone material such as a bioceramic material or bone tissue or containing bone tissue in combination with other biointegration enhancing components. The second material is incorporated in accessible voids such as open cells, pores, bore, holes and/or of the first component. The first component forms a frame or matrix for the second component. The first component imparts strength to the second component. The first and second components can receive one or more pharmaceutical substances. The second component can fully or partially disintegrate upon completion of the implanting to promote penetration of freshly grown bone tissue into the first component.

U.S. Pat. No. 5,716,416 to Lin discloses insertion of an elastic intervertebral implant.

U.S. Pat. No. 5,720,751 discloses spinal insertion tools including a tool with opposing implant engaging portions and including a pusher assembly. In one embodiment the implant engaging portions are fixed and in other embodiments the insertion portion is formed of two arms secured in scissors-like fashion. A pusher may include a threaded stem for attachment to the handle for advancement of the pusher bar toward and away from the implant by rotation of the threaded stem.

U.S. Pat. No. 5,741,253 to Michelson, discloses a threaded self tapping spinal implant and insertion instrumentation. The implant is tubular and cylindrical and is inserted in an opening in the spine formed by a drill inserted in a sleeve.

U.S. Pat. No. 5,443,514 to Steffee discloses an instrument for holding and inserting an inert spinal implant and which includes an intermediate portion, a handle and a clamp portion. The implant is wedge shaped with two opposing flat parallel surfaces and two inclined surfaces with vertebrae gripping ridges and which converge toward one end. The flat surfaces have recesses which receive the clamp of the instrument. The clamp comprises clamp halves with outwardly tapering surfaces and extensions which are received in the recesses. The extensions engage the flat bottom surfaces of the recesses. The clamp halves are drawn into mating inclined surfaces of the intermediate portion to force the clamp extensions against the implant recess bottom surfaces to compress the extensions against the implant. The insertion tool rotates the implant after it is inserted between adjacent vertebrae.

U.S. Pat. No. 5,782,830 to Farris discloses an implant insertion tool somewhat similar to the Steffee disclosure in that a pair of articulating jaws clamp an implant therebetween. The jaws are drawn together by forcing two resiliently mounted arms attached to the jaws into a tapered sleeve by displacing the sleeve along and relative to the arms.

U.S. Pat. No. 4,997,432 to Keller discloses an implant insertion instrument set which includes a vertebrae spreading instrument which includes two stop plates cooperating with two vertebrae spreading jaws forming a U-shaped recess. The jaws are shown offset at an angle to the handle longitudinal axis. A mechanism is between the jaws and handles which are spread apart by springs and locked together by a ratchet mechanism. The jaws are spread apart or drawn together by a screw drawing the jaws having beveled surfaces into or out of a beveled tube.

U.S. Pat. No. 6,174,311 to Branch discloses a C-shaped bone implant and implant holder tool for the PLIF approach. The tool has a pair of jaws for gripping the implant. In another embodiment, the holder has a threaded rod for holding the implant.

U.S. Pats. Nos. 5,885, 299, 5,885,300, 5,910,141, 6,004,326, 6,033,405, 6,042,582 and 6,063,088 illustrate still other insertion tools for a spinal implant.

U.S. Pat. No. 5,192,327 to Brantigan discloses oval and hemi-oval inert spinal implants which may be stacked together on mating ridges.

U.S. Pat. No. 5,814,084 to Grivas discloses a diaphysial cortical dowel implant which is generally circular cylindrical tapered at one end and having the natural intra-medullary canal passing therethrough. The dowel is obtained by a transverse cut of in the diaphysis of a long bone.

U.S. Pat. No. 5,865,845 to Thalgott discloses a metal spinal implant comprising a ring shaped body having opposed parallel sides spaced from a second pair of parallel sides. Upper and lower surfaces have teeth for engaging adjacent vertebrae. The implant has an interior space filled with hydroxyapatite, a ceramic material to promote bone growth.

U.S. Pat. No. 6,111,164 discloses a bone dowel similar in shape to that disclosed in the Grivas patent noted above. The dowel is cortical bone and free of extraneous cancellous bone not from the patient. Disclosed are femur, tibia and humerous bones from which the dowel may be formed.

U.S. Pat. No. 6,143,033 to Paul discloses an allogenic intervertebral implant which is an annular wedge shaped implant with a hollow core and teeth in a two dimensional array on opposing surfaces to engage opposing vertebrae.

ALIF implants have special problems not present in PLIF implants. These implants may use femoral rings as the access to the disc space is larger than the access space for the PLIF procedure. Space limitations inherent in the PLIF procedure often necessitates the use of spaced side-by-side implants as shown in several of the prior art patents noted above. Femoral rings made of cortical bone have different problems for insertion. The PLIF insertion tools typically have insertion load bearing surfaces that are adapted to apply insertion loads to the posterior end of the implant. Insertion loads and/or forces are defined herein as any type of force applied to the inserter and/or implant that tend to cause the implant and/or the inserter to move in the desired direction of insertion. Insertion loads and/or forces as used herein are defined as variable static, constant static, quasi-static and/or dynamic impact types of forces. Impact forces may be imparted by slap hammers for example. Insertion forces/loads as recognized by the present inventors do not necessarily have to be aligned in purely the direction of insertion, but must have a component in this direction.

When the implant is made of bone, it is relatively fragile. The insertion load application location on the prior art PLIF implants typically is on the posterior end of the implant. The implant has a longitudinal axis along which bone is present between the anterior and posterior ends in the axial direction of the applied insertion forces. For example, in the Grivas implant the posterior end is flat and extends across the implant so that axially directed forces are located across the implant including locations at which there is bone extending from the anterior to posterior ends.

To insert the implant, tools are required to not only grip the implant and readily release the implant after insertion but also are required to exert an insertion force on the implant during insertion. Such forces are typically applied in the prior art to a distal end surface of the implant as illustrated in several of the aforementioned prior art patents.

Femoral rings which are made of cortical bone, have a generally cylindrical outer peripheral surface and a central opening formed by the medullary canal and are generally too large for the posterior approach. Some rings may use the natural canal and others may have a canal that is altered to remove cancellous bone or is smoothed. As recognized by the present inventors, if insertion loads are applied by a flat insertion tool, such as a bone tamp, along the anterior-posterior central axis, the rings may be too weak for use with such tools due to the reduced ring cross section caused by the medullary canal along this axis. Such tools would apply insertion loads to the ring centrally along the insertion axis running substantially through the medullary canal. The bone at this location would be subjected to large bending and shear loads and may fracture if loads were to be applied at this location.

Thus a tool as shown in FIG. 3 of Michelson U.S. Pat. No. 5,522,899 noted above might be desirable except it has undesirable features for use with a bone ring. This tool has a curved surface for engaging a like surface of the implant. The problem with this tool for use with a bone ring implant is that it also uses a centrally located rib that mates with a centrally located channel in the implant edge surface abutting the tool. The channel creates a thinner cross section of a ring implant by reducing the cross section of the ring at that location. Further, a threaded hole is used in the implant to receive a threaded stud on the insertion tool. Such a groove and threaded hole are used to hold the implant and are not desirable for a femoral ring implant made of bone as the groove and hole reduce the amount of bone at that location and weaken the implant at that location. The curved surface of the tool while useful for applying insertion loads to the implant, does not provide a holding grip on the implant. Further, the implant described is made of metal, is of relatively high strength and thus does not have the problems associated with a ring implant made of bone.

None of the above patents or applications address or recognize a problem with insertion of an implant fabricated as discussed above. The present invention is a recognition of these problems with the insertion of an implant and is directed to provide a solution.

An implant according to one aspect of the present invention is for fusing and/or supporting bone of a human or animal defining an implant receiving space and defining anterior and posterior positions with respect to the recipient implant site. The implant comprises a body having a peripheral outer surface formed by at least one peripheral side wall and opposing top and bottom surfaces, the top and bottom surfaces for engaging adjacent bone of said implant receiving space, the body having an anterior end and a posterior end defining an anterior/posterior axis corresponding to the recipient implant site respective anterior and posterior positions, the axis defining a plane between the top and bottom surfaces that is approximately equidistant from the top and bottom surfaces.

The body exhibits different degrees of strength in corresponding different peripheral regions in respect of an insertion force applied to the body in the plane in an insertion direction for inserting the body into the implant receiving space, at least one of the different peripheral regions being the weakest in respect of the insertion force.

The at least one side wall has at least one recess located at a peripheral region exhibiting a strength in the plane in the insertion direction greater than the at least one weakest region for receiving the insertion force to thereby minimize damage to the body during the insertion.

In one aspect, the insertion force defines an implant insertion axis, the body having a gripping first surface for receiving a body insertion gripping force applied to the body in a direction generally normal to the insertion axis.

In a further aspect, the body is bone, preferably cortical bone, and more preferably formed by a transverse slice of the diaphysis of a long bone.

Preferably, the implant is for use in fusing vertebrae.

An implant according to a further aspect of the present invention is for fusing and/or supporting bone of a human or animal defining an implant receiving space and defining anterior and posterior positions with respect to the recipient implant receiving space, the implant for insertion into the implant receiving space in an insertion direction. The implant comprises a body having opposing top and bottom surfaces and a peripheral outer surface intermediate the top and bottom surfaces, the top and bottom surfaces for engaging bone of the implant receiving space, the body having an anterior end and a posterior end defining an anterior/posterior axis corresponding to the implant receiving space respective anterior and posterior positions.

The peripheral outer surface has at least one recess having a first surface for receiving a body gripping force transverse to the implant insertion direction and a second insertion load receiving surface transverse to the first surface and transverse to the implant insertion direction for insertion of the body into the implant receiving space in the insertion direction.

In a further aspect, the peripheral outer surface has a planar surface at and defining the anterior end and the at least one recess is spaced from the planar surface.

In a further aspect, the at least one recess is located on the body for insertion of the body in a direction transverse to the anterior/posterior axis of the vertebral bone. In a further aspect, the at least one recess is located on the body for being gripped and inserted in an insertion direction in the range of about 0° to about 90° to the anterior/posterior axis.

In a further aspect, the body has regions of differing strengths such that an insertion load at the weaker region will damage the body, the at least one recess being located at a body region which will minimize damage to the body during insertion.

In a further aspect, the body has a generally central chamber, the at least one recess being axially aligned on an axis passing through the body on a side wall between the chamber and the outer peripheral surface.

In a further aspect, a pair of recesses are aligned on a corresponding axis passing through the body at opposite sides of the chamber.

In a further aspect, the recess first surface is generally aligned in the insertion direction with a portion of the body on a side of the chamber.

In a further aspect, the first surface is arcuate. In a still further aspect, the gripping first surface is curved. In a further aspect, the gripping first surface is planar and the second surface is planar transverse to the first surface. In a still further aspect, a plurality of recesses are provided and may be identical or different. In a still further aspect, the recesses are of the same shape, but different dimensions.

In a further aspect, at least one of the recesses is in communication with the top and/or bottom surfaces of the implant.

In a further aspect, the implant has an annular peripheral surface, the peripheral surface having a planar surface at and defining the anterior end.

In a further aspect, the at least one recess is located on the implant for insertion in a direction transverse to the anterior/posterior direction of the bone to be fused and/or supported.

In a further aspect, the at least one recess is located on the implant for gripping and receiving insertion loads applied by the insertion tool jaw in an insertion direction in the range of about 0° to about 90° to the anterior/posterior direction.

In a further aspect, the body further includes a pair of the recesses aligned on a corresponding axis passing through the implant on opposite sides of a chamber.

In a further aspect, the body is C-shaped, the body having top and bottom surfaces, a first peripheral side wall surface between the top and bottom surfaces extending between anterior and posterior ends and a second peripheral side wall surface opposite the first side wall surface extending between the ends and between the top and bottom wall surfaces, the second side wall surface being defined by first and second planar surfaces interrupted by an intermediate concave surface, the at least one recess being located in the first peripheral side wall surface.

In a further aspect, the at least one recess is located generally adjacent to the posterior end.

In a further aspect, the first planar surface is adjacent to the anterior end of the implant and the second planar surface is adjacent to the posterior end of the implant, the implant including a further recess in the first planar surface, the further recess having a gripping surface for cooperating with the at least one recess and a surface for receiving an insertion force imposed on the at least one recess for insertion of the implant.

In a further aspect, an insertion tool is provided for holding and inserting an implant in an insertion direction for fusing and/or supporting bone and comprises first and second jaws movable in implant gripping and release directions respectively toward and away from each other, each jaw having a first implant gripping surface for gripping the implant, the first jaw for gripping the implant first gripping surface and having a tip surface at the terminal end of the first jaw distal the mechanism means set forth below, the tip surface for engaging the implant second surface for the insertion of the implant with an insertion load relative to the bone for the fusing and/or supporting the bone. Mechanism means manually move the jaws in the directions of implant gripping or releasing.

In one aspect, the mechanism means comprises first and second arms movably secured relative to each other and terminating at first ends distal the jaws, resilient means for resiliently biasing the arms apart in a implant release position and holding means for holding the arms against the bias of the resilient means in a implant gripping position, the second jaw having a implant gripping surface for gripping the implant in cooperation with the first jaw for holding the implant during insertion of the implant.

In a further aspect, each of the arms includes a first arm portion extending transverse to that arm, the arm first portions having at least a further portion, the further portions overlapping.

In a further aspect, at least one of the arm first portions is arranged for receiving an insertion force for driving the implant into a spinal disc space.

In a further aspect, one of the first and second jaws has a planar implant gripping surface and the other of the first and second jaws has a non-planar implant gripping surface.

In a still further aspect, the non-planar implant gripping surface of the other jaw is arranged to tangentially abut the implant first surface.

In a further aspect, the non-planar surface is curved.

In a still further aspect, each of the arms has a longitudinal axis, each arm first portions being transverse to that arm longitudinal axis, the one arm first portion being joined to its arm by a stop for limiting closing relative displacement of the other arm first portion.

In a further aspect, the mechanism means comprises first and second arms pivotally secured together and terminating at first ends distal the jaws, resilient means resiliently biasing the arms apart in an implant release position and holding means for holding the arms against the bias of the resilient means in an implant holding position.

In a further aspect, each arm includes a first arm portion extending transverse to that arm, the arm first portions having at least a further portion, the further portions overlapping.

In a further aspect, at least one of the arm first portions is arranged for receiving an insertion force for driving the implant into a disc space.

In a still further aspect at least one of the jaws has a non-planar implant gripping surface.

In a further aspect, the non-planar implant gripping surface of the jaw is curved and may be complementary to the implant first surface configuration in one aspect or in a further aspect may contact the implant tangentially at an implant gripping surface.

In still further aspect, the mechanism comprises a tubular housing; a jaw member in the housing having a threaded bore at a first end and first and second arms extending toward a second end opposite the first end, the arms extending beyond the housing and each terminating in a respective jaw, each arm being resilient relative to the first end; a rod in the housing threaded to the threaded bore at a first rod end and terminating in a projection at a second rod end distal the rod first end, the housing having a recess adjacent to the projection; and a knob for mating with the recess and for mating with the projection for rotating the rod relative to the jaw member to thereby displace the jaw member relative to the housing axially along the housing, the housing and arms being arranged to selectively open and close the jaws in response to the relative axial displacement of the jaw member to the housing.

In a further aspect, first and second jaws are movable in directions respectively toward and away from each other, each jaw having a tip surface at the terminal end of the first and second jaws distal the mechanism means set forth below, the tip surface for engaging the implant first insertion load bearing surface for insertion of the implant relative to the bone for the fusing or supporting. Mechanism means manually move the jaws in the gripping or releasing directions. A rod is secured to the mechanism means for releasably holding the implant in a position for engagement by the jaws.

### IN THE DRAWING

FIGS. 1, 5, 8, 11 and 14 are plan views of cortical bone spinal implants according to different embodiments of the present invention;

FIGS. 2, 6, 9, 12 and 15 are respective side elevation views of the spinal implants according to the different embodiments of FIGS. 1, 5, 8,11 and 14;

FIGS. 4, 7, 10, 13 and 16 are respective anterior end elevation views of the implants according to the different embodiments of FIGS. 1, 5, 8, 11 and 14;

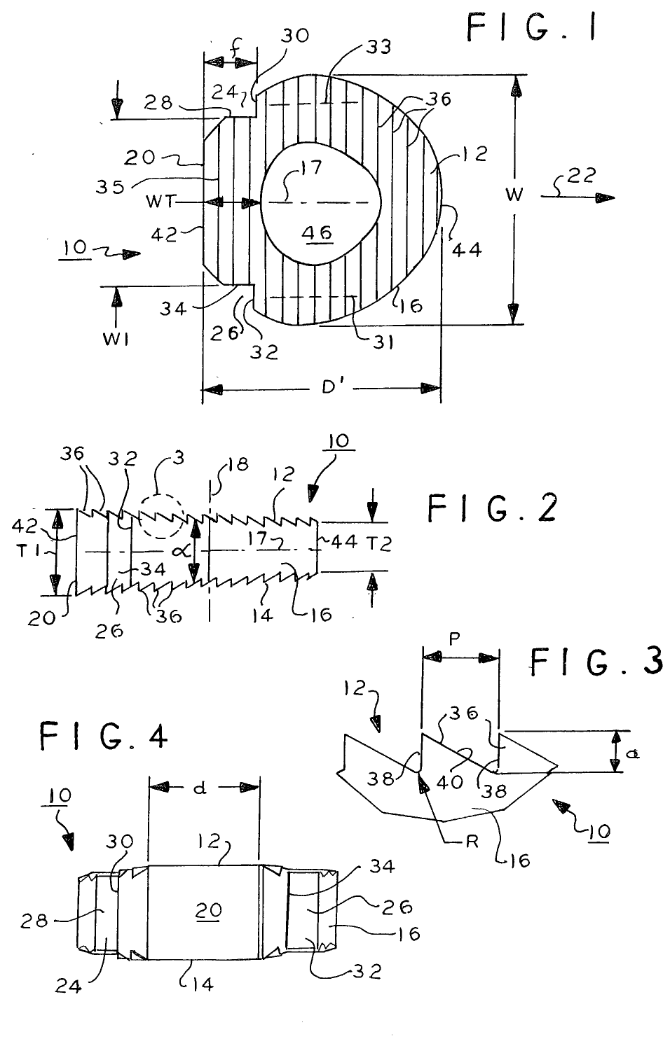

FIG. 3 is a side elevation more detailed representative view of the implant of FIG. 2 taken at region 3;

FIG. 17 is an isometric view of an implant insertion tool according to one embodiment;

FIG. 18 is a plan view of the tool of FIG. 17 with the implant of FIGS. 14-16 and 20 being held thereby;

FIG. 19 is an isometric view of an implant gripping jaw of the tool of FIG. 18;

FIG. 19a is an isometric view of an alternative embodiment of an implant gripping jaw;

FIG. 19b is an end sectional view of implant 116 of FIG. 14 recess 118 being gripped by the jaw of FIG. 19a;

FIG. 20 is an isometric view of the implant of FIGS. 14-16;

FIG. 21 is an isometric view of an implant insertion tool according to a second embodiment;

FIG. 22 is a side elevation sectional view of the tool of FIG. 21;

FIG. 23 is a more detailed sectional view similar to the view of FIG. 22;

FIG. 24 is a fragmented isometric view of the implant gripping jaws of the implant insertion tool gripping an implant similar to the view of FIG. 21;

FIG. 25 is a side elevation view of the tool of FIG. 24;

FIGS. 26 and 27 are respective fragment side elevation and plan sectional views of a representative human spine;

FIG. 28 is an isometric view of an anterior approach implant insertion tool according to a further embodiment;

FIG. 29 is an isometric exploded view of the tool of FIG. 28;

FIG. 30 is a side elevation view of a shaft of the tool of FIGS. 28 and 29;

FIGS. 31 and 32 are respective top plan and side elevation views of the implant gripping jaws of the tool of FIGS. 28 and 29;

FIG. 33 is an end elevation view of the jaws of FIG. 31;

FIG. 34 is a more detailed fragmented side elevation view of a jaw of the tool of FIG. 31 taken at region 34;

FIG. 35 is a sectional elevation view of the tubular outer housing of the tool of FIG. 28;

FIG. 36 is a side elevation partially in section view of a thumb screw for use with the tool of FIGS. 28 and 29;

FIG. 37 is an isometric view of a ring implant according to a further embodiment;

FIGS. 38, 39 and 40 are respective sectional plan views of the implant of FIG. 37 taken along lines 38-38 of FIG. 40, an anterior view and a side elevation view taken along lines 40-40 of FIG. 38;

FIG. 41 is a sectional plan view of the implant of FIG. 42 taken along lines 4141;

FIG. 42 is a side elevation view of the implant of FIG. 41;

FIG. 43 is an anterior/lateral side elevation view of the implant of FIG. 42;

FIG. 44 is an anterior side elevation view of the implant of FIG. 42;

FIG. 45 is a sectional plan view of the implant of FIG. 46 taken along lines 4545;

FIG. 46 is a side elevation view of the implant of FIG. 47;

FIG. 47 is an isometric view of an implant according to a further embodiment;

FIG. 48 is an anterior view of the implant of FIG. 46;

FIG. 49 is an isometric view of a ring implant according to a further embodiment;

FIGS. 50, 51 and 52 are respective side elevation view of the implant of FIG. 49, a sectional plan view of the implant of FIG. 50 taken along lines 51-51, and an anterior elevation view of the implant of FIG. 49;

FIGS. 53-56 are a respective isometric view of an implant according to a further embodiment, a side elevation view, a plan sectional view taken along lines 55-55 of FIG. 54 and anterior elevation view of the implant of FIG. 53;

FIG. 57 is an isometric view of the implant of FIG. 53 being gripped by the jaws of the tool of FIG. 58 taken at region 57;

FIG. 58 is an isometric view of an implant insertion tool according to a further embodiment gripping the implant of FIG. 53;

FIG. 59 is a more detailed isometric view of the implant gripping jaws of the tool of FIGS. 57 and 58;

FIG. 60 is an isometric exploded view of the tool of FIG. 58;

FIG. 61 is an end elevation view of a portion of an implant and insertion tool jaw gripping the implant in the implant gripping recess;

FIG. 62 is a diagrammatic isometric illustration of an implant according to a further embodiment;

FIG. 63 is an isometric view of an implant according to a further embodiment;

FIG. 64 is a side elevation of the implant of FIG. 63;

FIG. 65 is a sectional plan view of the implant of FIG. 64 taken along lines 65-65;

FIG. 66 is a side elevation view of the implant of FIG. 63 taken along lines 66-66;

FIG. 67 is an isometric view of the tool of FIGS. 58 and 60 with modified jaws for gripping the implant of FIGS. 63-66;

FIG. 68 is an isometric view of an insertion tool according to a further embodiment;

FIGS. 69-71 are respective plan, end and side elevation views of an implant according to a further embodiment;

FIG. 72 is an isometric view of a recess of the implant of FIG. 67;

FIG. 73 is a fragmented plan view of an insertion tool and the implant of FIG. 67 in an insertion mode;

FIG. 74 is an isometric view of the insertion tool jaws of the tool of FIG. 73;

FIGS. 75, 76 and 77 are respective plan, side and end elevation views of an implant according to a further embodiment;

FIG. 78 is an isometric view of the jaws of an implant insertion tool according to a further embodiment of the present invention;

FIG. 79 is an isometric view of one of the insertion tool jaws of the tool of FIG. 78 shown in more detail;

FIG. 80 is an end elevation view of the insertion tool jaw of FIG. 79;

FIG. 81 is a top plan view of the insertion tool jaw of FIG. 79;

FIG. 82 is a sectional elevation view of the insertion tool jaw of FIG. 81 taken along lines 82-82;

FIG. 83 is an isometric view of a bone spinal implant inserted by the tool of FIGS. 78-82;

FIG. 84 is a top plan view of the implant of FIG. 83;

FIG. 85 is an anterior end elevation view of the implant of FIG. 83;

FIG. 86 is a side elevation view of the implant of FIG. 83; and

FIG. 87 is a sectional plan view of the implant of FIG. 86 taken along lines 86-86.

The intervertebral wedge shaped implant 10, FIG. 1, which is also referred to as a plug, a graft, and sometimes referred to as a ramp when wedge shaped, is preferably made of bone, more preferably relatively hard cortical bone, or, in the alternative, it may be any other known bone or synthetic or other biocompatible material such as titanium, cancellous bone or combination thereof, or other materials used for implants such as metals, polymers, xenografts, composites, bone containing composites and so on. The implant 10 has a top surface 12, a bottom surface 14 and a side peripheral surface 16 extending between the top and bottom surfaces. The outer peripheral surface 16 is generally irregular and somewhat oval and is defined by a straight line that is normal to the axis 17 and parallel to axis 18 and moved in translation about the axis 18 to form the contour of FIG. 1. This contour is usually, but not limited to, that of the donor bone from which the implant harvested. Thus this contour will vary from implant to implant based on the donor bone outer surface configuration from which the implant is harvested. The implant shown may be a femoral ring when harvested from the femur bone of a donor. The implant may be harvested from the tibia or other bones of a donor according to a given implementation as known in this art. The outer peripheral surface 16 thus is, in this embodiment, normal to the plane of the drawing sheet as best seen in FIGS. 2 and 4. The peripheral surface 16 has an anterior planar end surface 20 that is machined from the donor bone and is generally normal to the insertion direction 22 in an anterior approach procedure. This surface 16 defines the anterior end of the implant to a surgeon.

The implant 10 has two recesses 24 and 26 formed in the peripheral surface 16. Generally the implant without the recesses is generally symmetrical relative to the axis 17, but may be asymmetrical in accordance with the bone from which the implant is harvested. The recess 24 has a first surface 28 parallel to axis 17 and a second surface 30 normal to surface 28 and parallel to surface 20 in this embodiment forming a right angle recess. Recess 26 is formed in surface 16 on side of the surface 16 opposite recess 24. Recess 26 has a surface 32 parallel to surface 30 and preferably aligned with surface 30. Recess 26 also has a surface 34 normal to surface 32.

Thus the recesses 24 and 26 are generally positioned in mirror image relation relative to the axis 17, although recesses 24 and 26 preferably are dimensioned differently. The surfaces 28 and 34 are arranged to receive mating gripping jaws such as by the tools of FIGS. 18-23 to be described below. The surfaces 30 and 32 are arranged to receive and abut the tips of the jaws of the tools of FIGS. 18-23 for receiving an insertion force imparted by the jaws to be described. The insertion force, may be a continuous pushing, i.e., a constant or variable static analog force, an impact or pulse force or a combination of different forces that may form a load on the implant, as mentioned in the introductory portion. The insertion force is directed primarily through the solid side walls (cortical bone for a cortical ring) of the implant at axes 31, 33, and not through the central portion 35 aligned with the medullary canal, bore 46. That is, the insertion forces are not directed in axial alignment with the medullary canal. The insertion forces are exerted along the side(s) of the implant so that the forces are exerted primarily on a solid bone portion along axes 31 and 33 extending from a peripheral side at the anterior end of the implant to the posterior end.

In this embodiment, the recesses 24 and 26 are dimensioned differently, but may be the same in other embodiments. The surfaces of the recesses 24 and 26 in this embodiment thus extend parallel to the axis 18 in communication with the respective top and bottom surfaces 12 and 14.

Surfaces 12 and 14 are preferably inclined at an angle a to form a wedge shaped implant, but also may be rectangular of uniform thickness. The relative angle a of the surfaces 12 and 14, FIG. 2, accommodates the inclination of the adjacent vertebrae to maintain the natural curvature of the spine and preferably could lie in the range of about 4°-10° and more preferably 80 in this embodiment. The implant 10 has a posterior end 44 and anterior end 42 at anterior end defining planar surface 20.

Respective top and bottom surfaces 12 and 14 both have an optional array of identical parallel teeth 36 formed by transversely extending grooves in the top and bottom surfaces. The teeth 36 form saw teeth and have a posterior facing rake 40 and an anterior facing rake 38. The teeth 36, FIG. 3, have a depth a of about 1 mm to the theoretical root intersection of tooth walls 38 and 40. The intersection is formed by a radius R preferably about 0.1 mm. Rake 38 is preferably normal to axis 17 (FIG. 2) and rake 40 is preferably inclined about 30° to axis 17. The tooth pitch P is preferably about 2 mm in this embodiment. The rakes 38 and 40 intersect at the tooth crests at a sharp edge that lie in planes normal to axis 17 and extend transversely linearly across the implant to opposing edges at opposite sides of the peripheral wall 16. The array of transverse teeth 36 preferably extends from anterior end 42 to posterior end 44. The teeth bite into the vertebrae after insertion into the disc space and the rakes are arranged to preclude the implant from backing out of the disc space once inserted. The spacing and dimensions of the teeth optimize the strength of the teeth 36 for this purpose.

In the alternative, the top and bottom surfaces 12 and 14 may have other forms of roughness to grip the adjacent vertebra such as cross cut teeth formed generally as pyramids, waffle shaped teeth or surfaces, knurlings, grooves, crisscross raised peaks and/or grooves, ridges, continuous or intermittent, dimples, recessed or raised, or other shapes of upstanding projections or recesses and/or grooves.

The top and bottom surfaces 12 and 14 are machined to form the desired configurations such as the preferred wedge shape taper of the implant 10, FIG. 2. Other shapes may also be provided as desired. The implant 10 has a transverse width W which preferably is in the range of 20-30 mm. The implant length dimension D parallel to axis 17 is preferably in the range of about 20-32 mm and more preferably may be in the range of about 24-27 mm for one size implant and in the range of about 28-30 mm for a second size implant. The implant 10 has a through bore 46 parallel to axis 18 formed by the medullary canal and thus will be dimensioned accordingly. The canal forming the bore 46 may also be machined if desired to other configurations. The recesses 24 and 26 are spaced apart dimension WI a distance of preferably about 6-20 mm. The depth f of the recesses 24 and 26 respective walls 30 and 32 from the anterior flat end wall surface 20 is preferably about 1-15 mm. The minimum thickness dimension WT between the bore 46 and the end surface 20 along the axis 17 is preferably about 3-7 mm. These dimensions of the recesses accommodate the insertion tool jaw configurations to be described below and are given by way of illustration only, and may vary as necessary.

The recesses 24 and 26 are located so that a projection of the recesses in direction 22 parallel passes primarily through a continuous section of bone terminating at an opposite location on the peripheral surface 16 in that direction. In this way an insertion force for inserting the implant into the intervertebral space is transmitted primarily through the side bone sections in direction 22. In comparison, if the insertion force were exerted on surface 20, the bone at minimum dimension WT is much shorter in direction 22 than the section aligned with the recesses. An insertion force at the center of the implant at surface 20 could tend to distort and/or damage the implant due to the presence of the bore 46 and the reduced thickness of the bone in direction 22 at this location. Therefore insertion forces at the recesses walls 30 and 32 in direction 22 has a sufficient amount of, as well as proper orientation of bone necessary to support the compressive loads generated during insertion.

The flat surface 20 is formed in the implant to provide a controlled length dimension D of the implant during fabrication, FIG. 1.

Preferably the implant is formed from cadaveric human or animal bone and/or bone composites of sufficient strength to support adjacent vertebra when fused thereto, and more preferably of a long human or animal bone and comprising primarily cortical bone, which is hard and exhibits relatively good strength. For example, see U.S. Pats. Nos. 5,899,939, 6,123,731, and 6,294,187 all incorporated by reference herein.

Preferably, the implant 10 is formed from the cortical ring of a long bone, such as the fibula, ulna, humerous, tibia or femur by cutting the bone transversely across the diaphysis or metaphisis of the bone. This forms a cortical ring. Typically, larger bones are used to form implants for thoracic and lumbar spinal fusion. Smaller bones including the ulna, radius and fibula are used to form implants for cervical spinal fusion. The cut bone is secured and the peripheral side wall machined as described to provide, in one embodiment, a substantially somewhat oval implant with a flat anterior surface 20 and the recesses 24 and 26.

Preferably, after the implant is formed, the bone is partially demineralized by placing it in a 0.6 Normal HCL solution. By demineralizing the implant, all of the peripheral surfaces of the implant will be demineralized. The strength of the implant will not substantially be compromised. Moreover, the bone may be treated using a variety of bone healing enhancing technologies. For example, bone growth factors may be infused into the natural porosity of the bone and/or the bone may be infused with acid to further demineralize the internal matrix of the bone. These treatments may be performed using the pressure flow system disclosed in U.S. Pat. No. 5,846,484 incorporated by reference herein or other known appropriate methods.

While human bones are preferred, non-human animal bones may also be used.

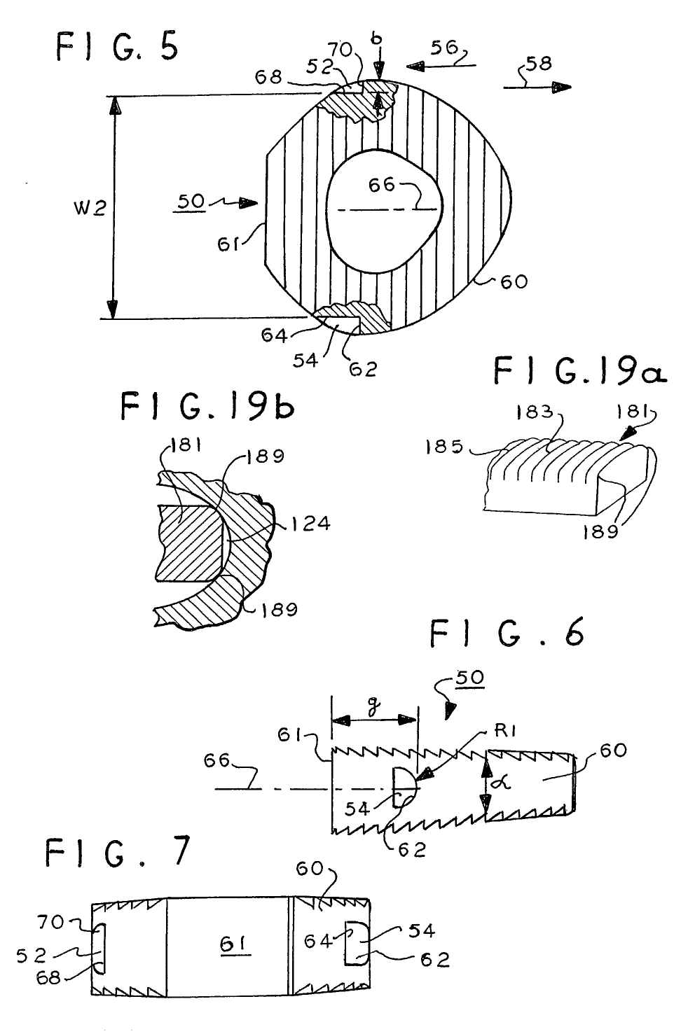

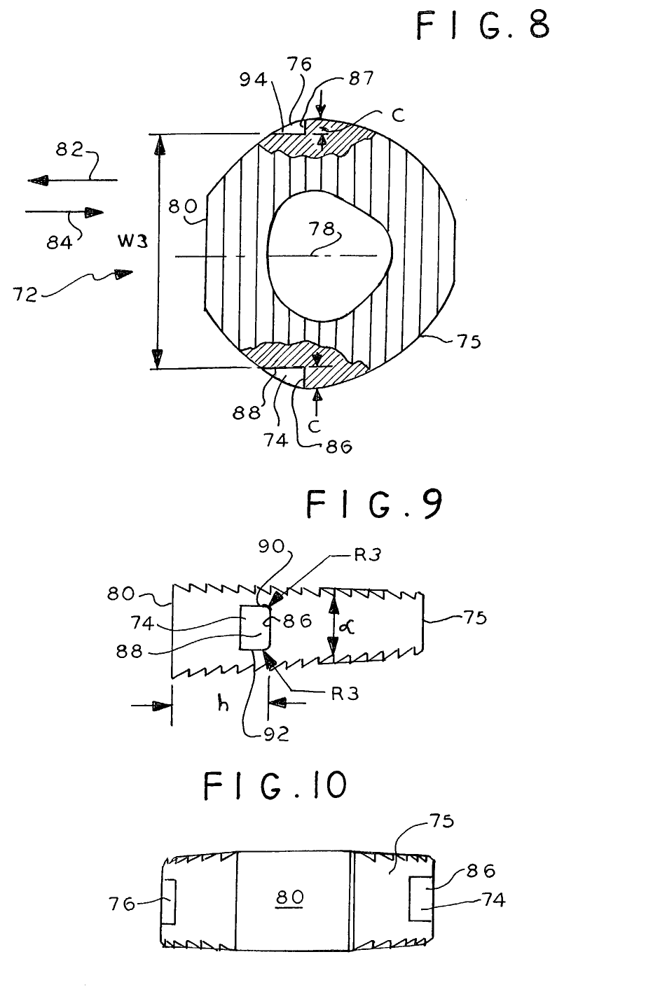

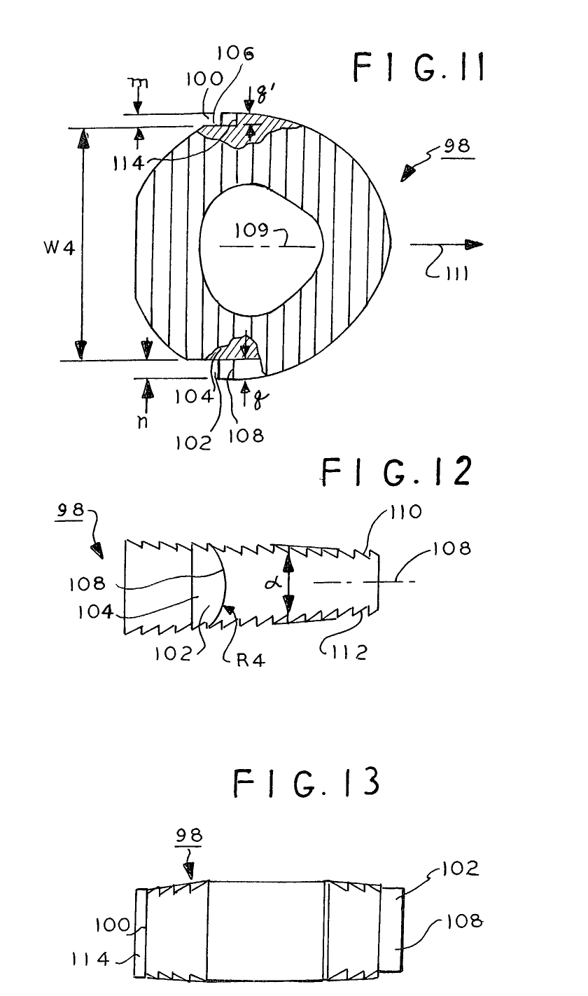

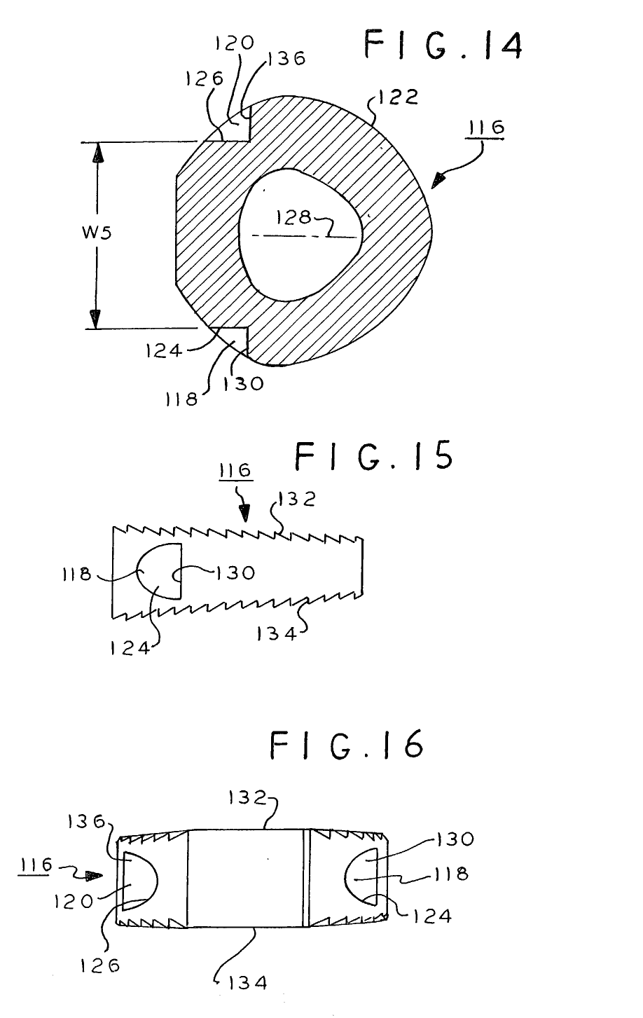

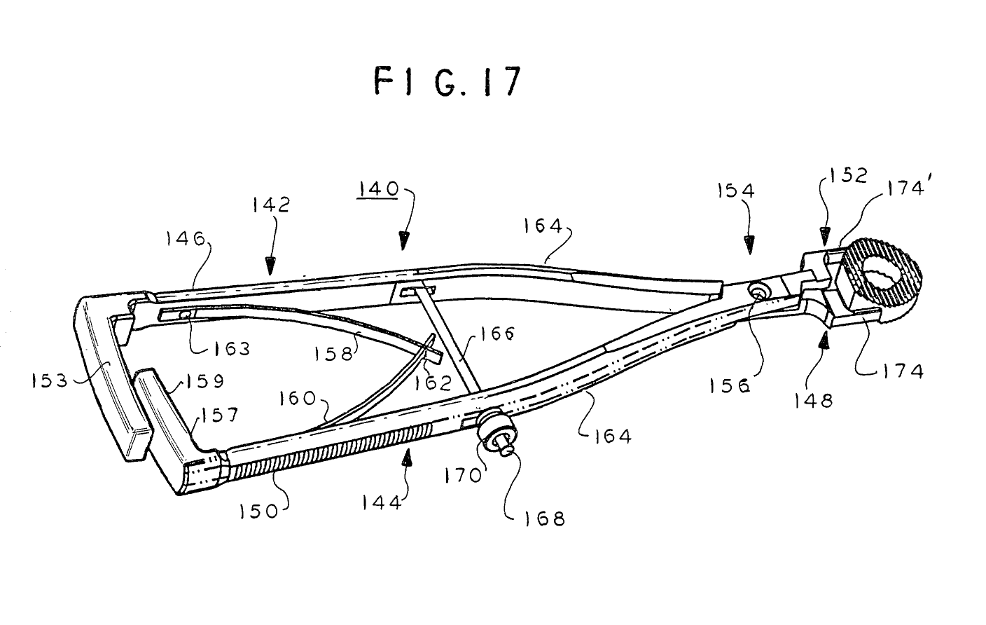

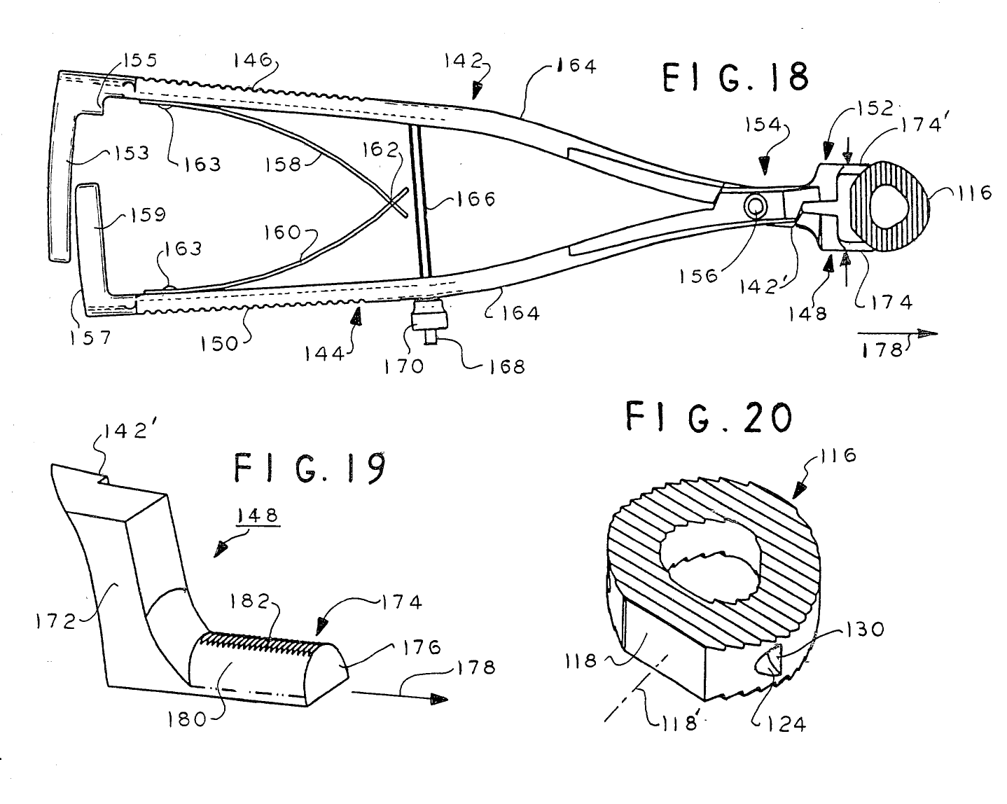

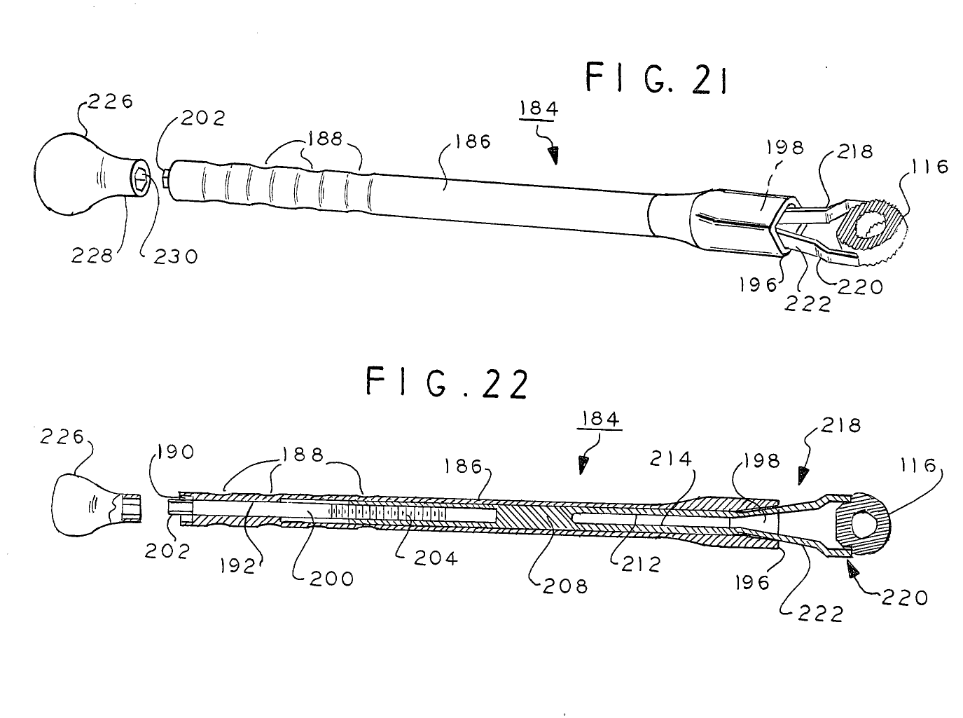

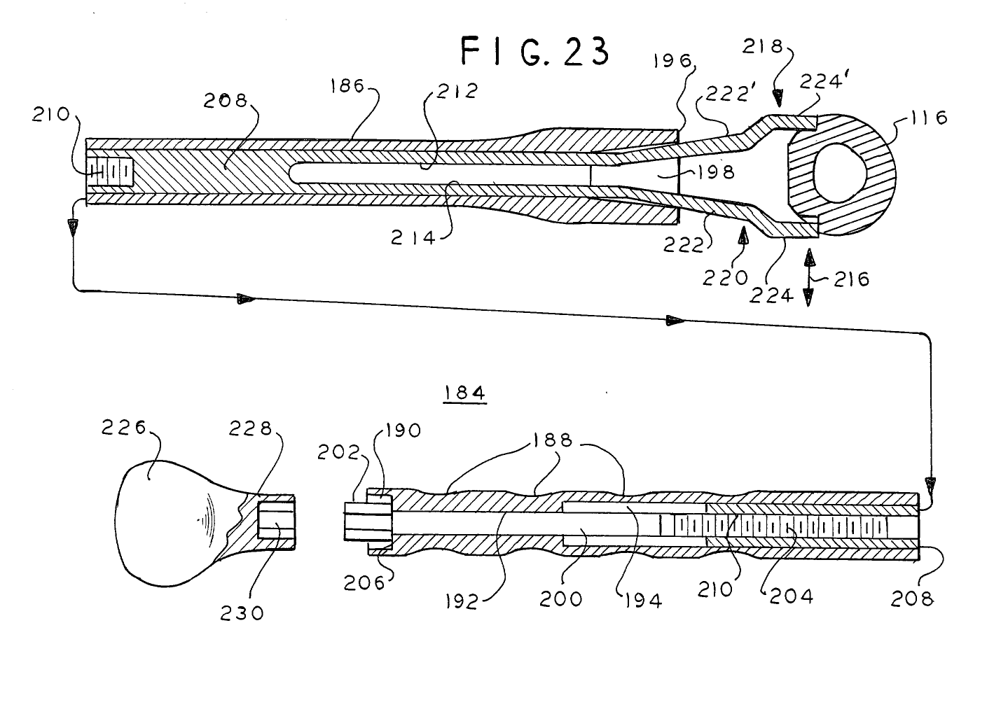

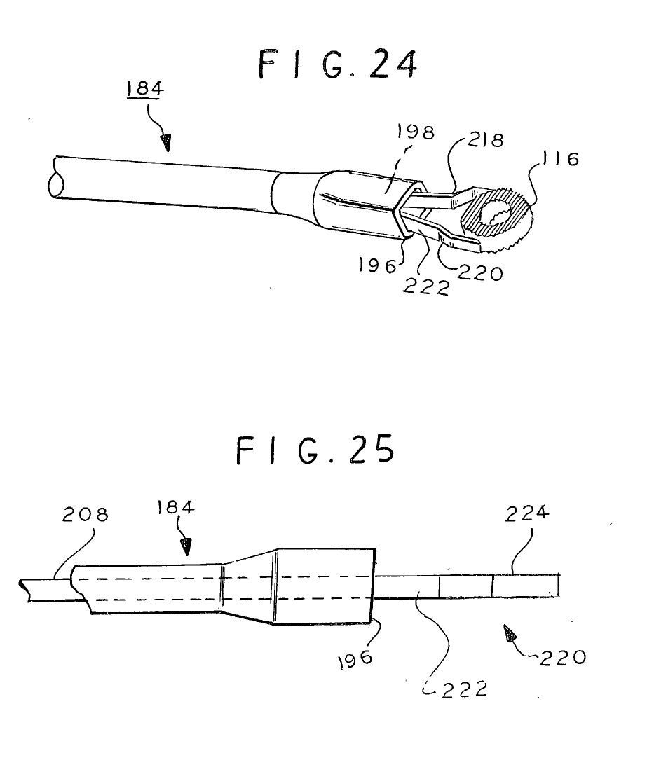

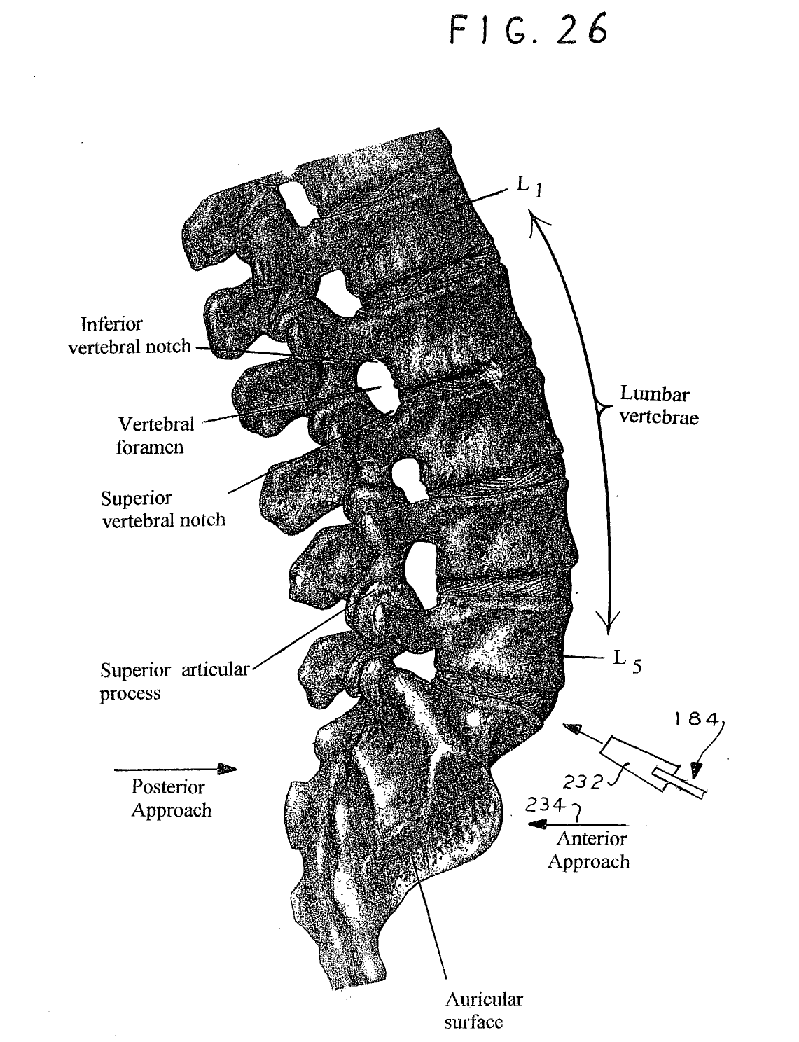

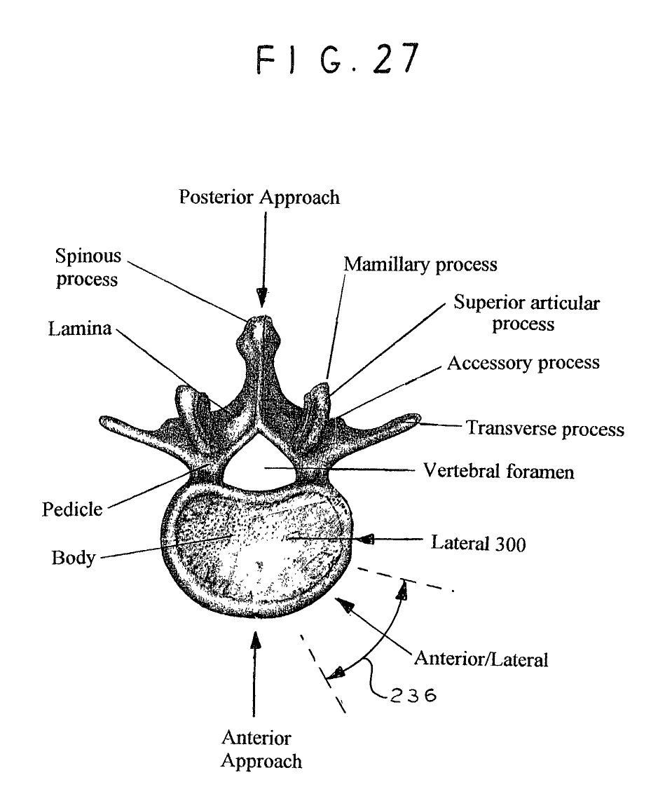

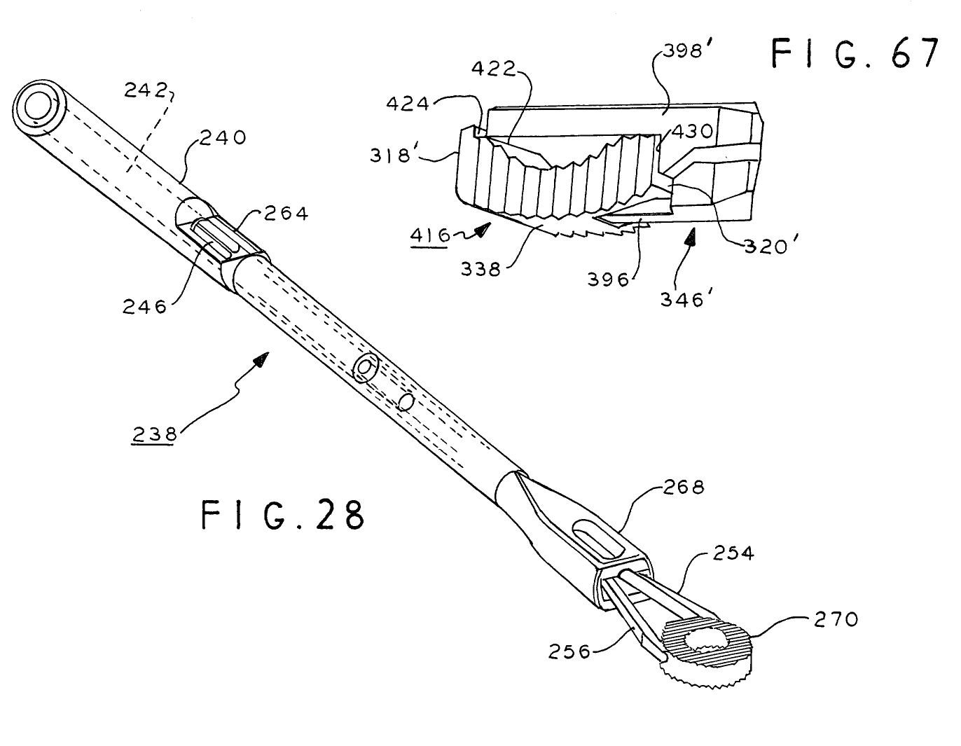

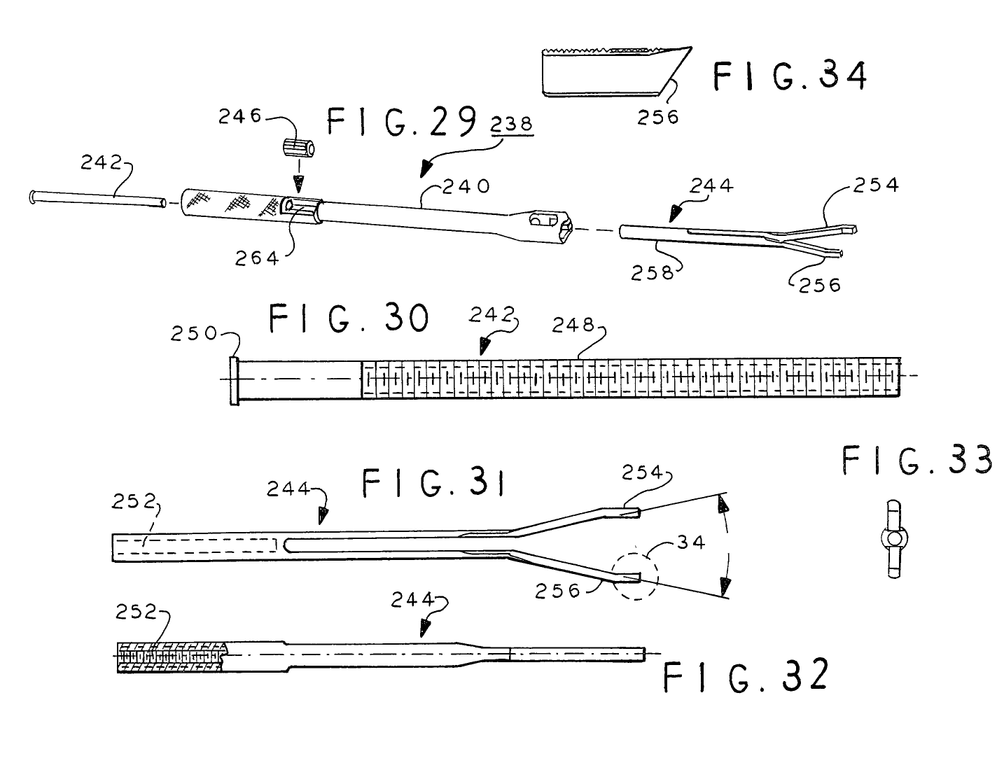

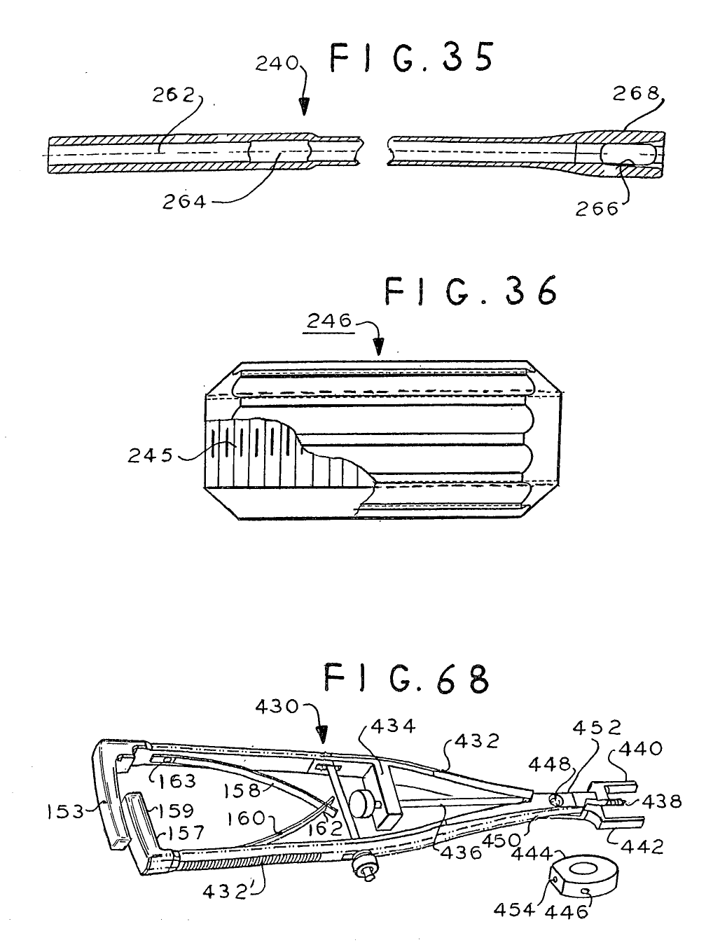

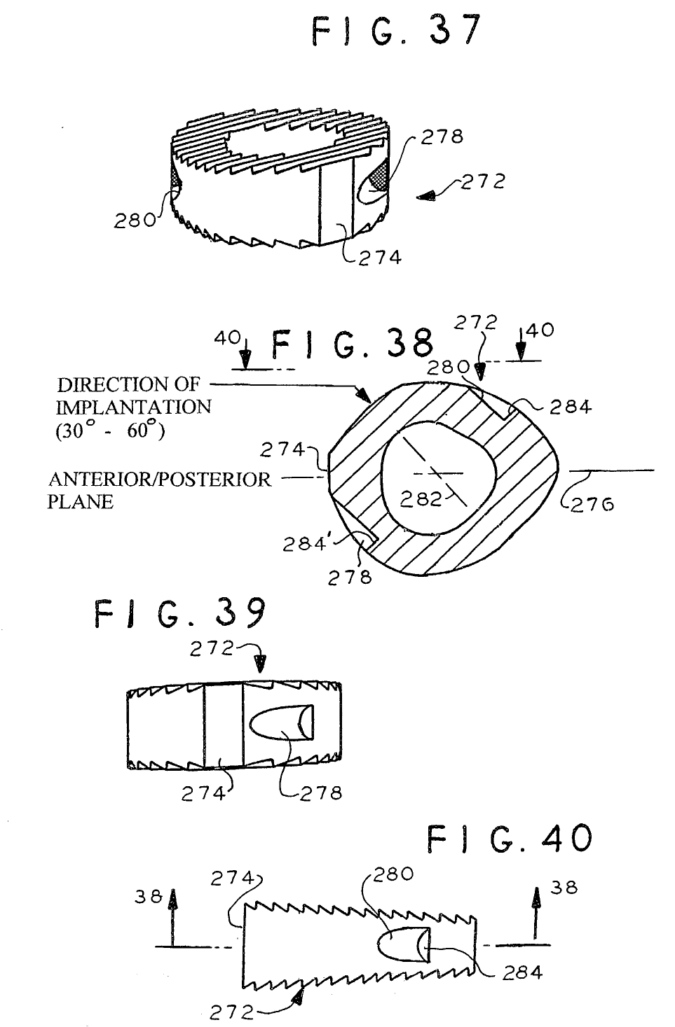

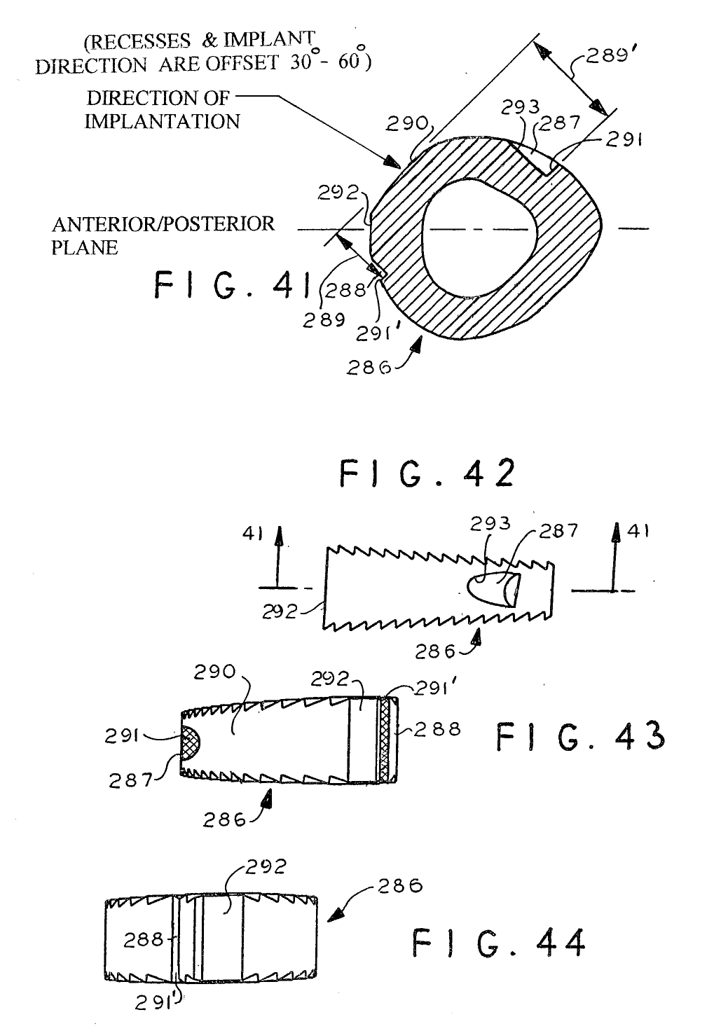

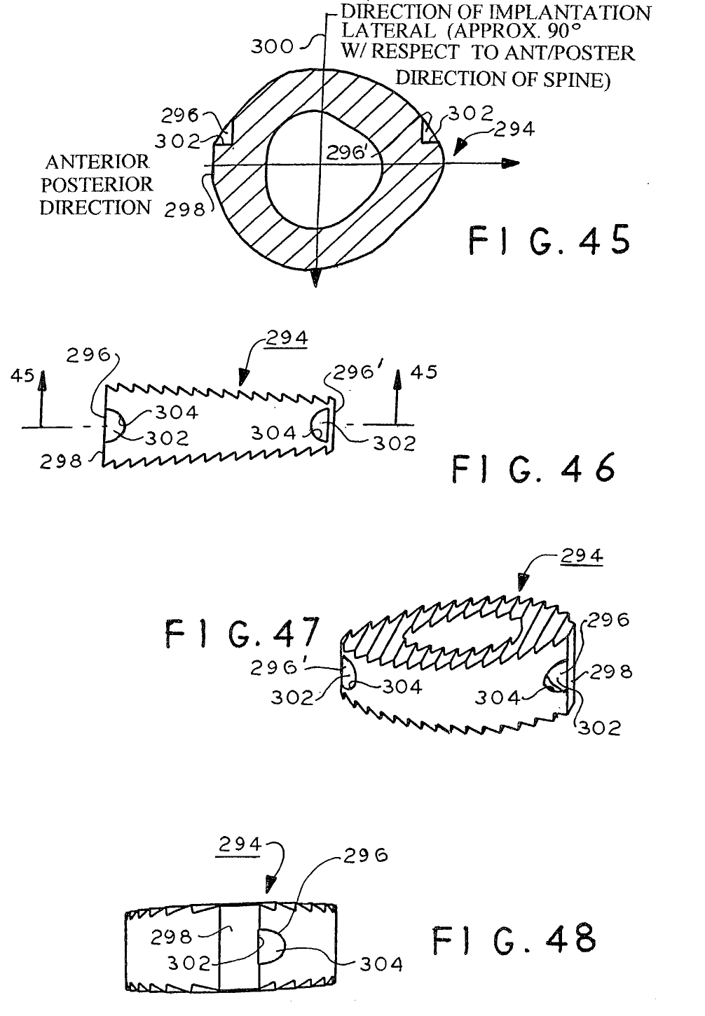

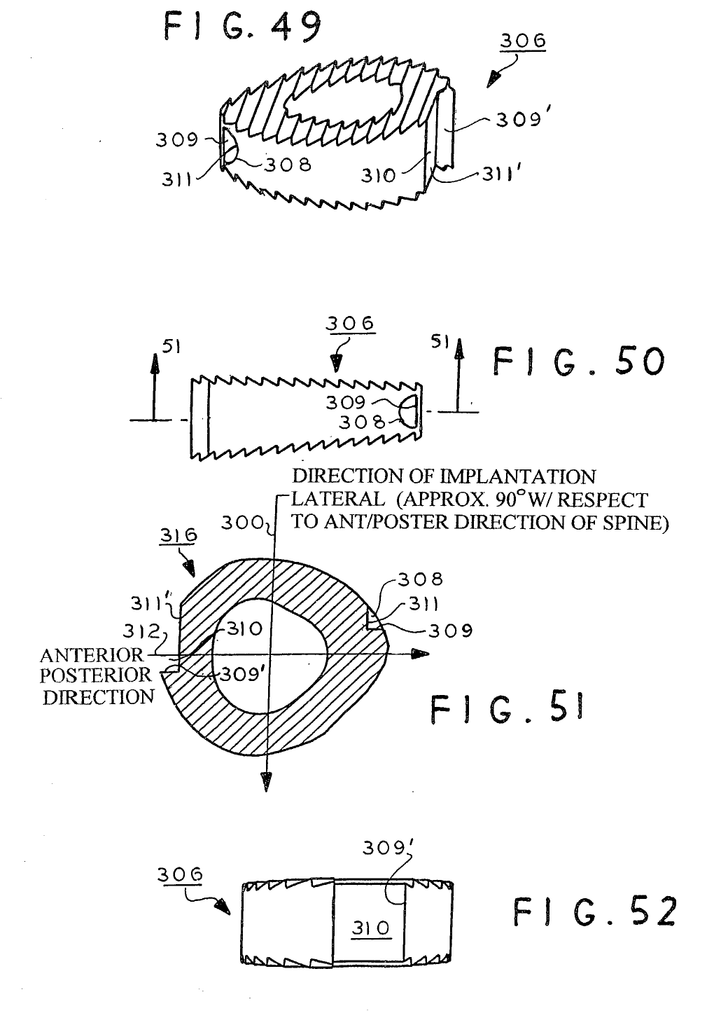

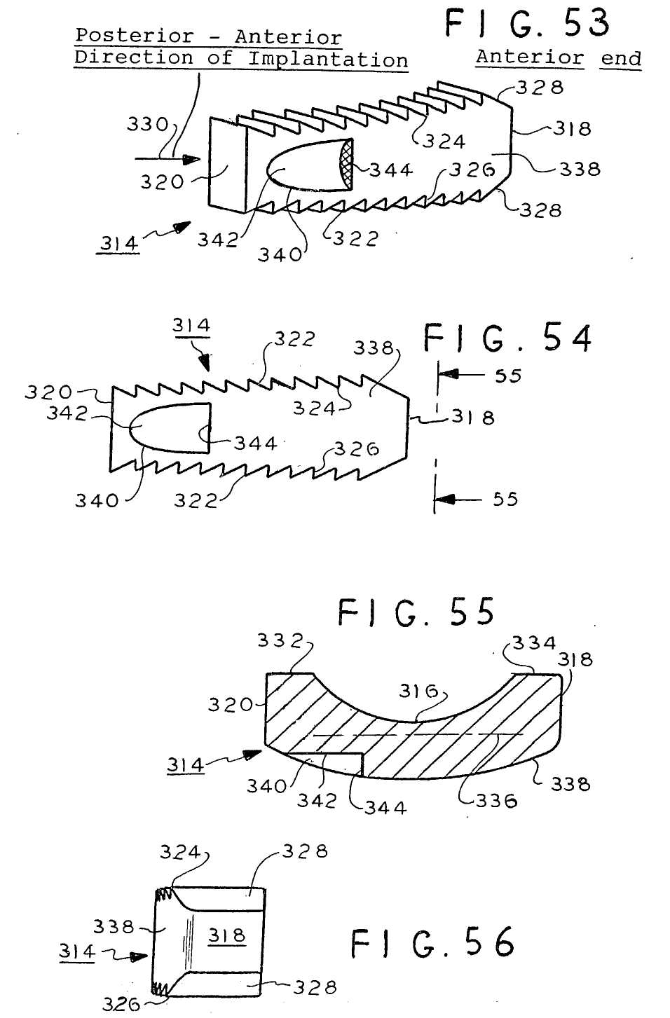

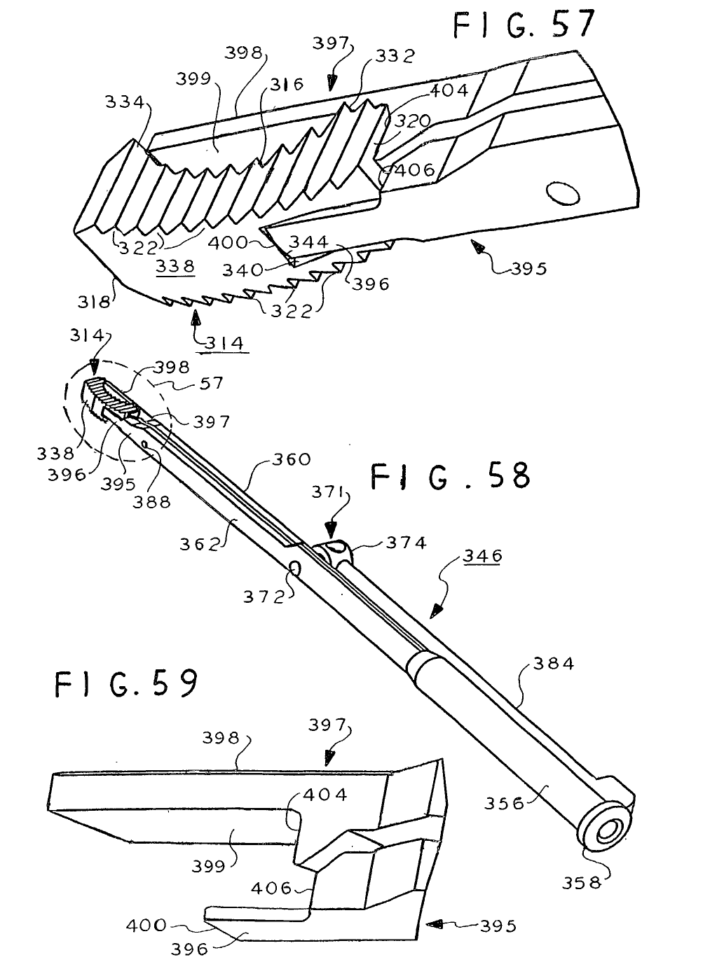

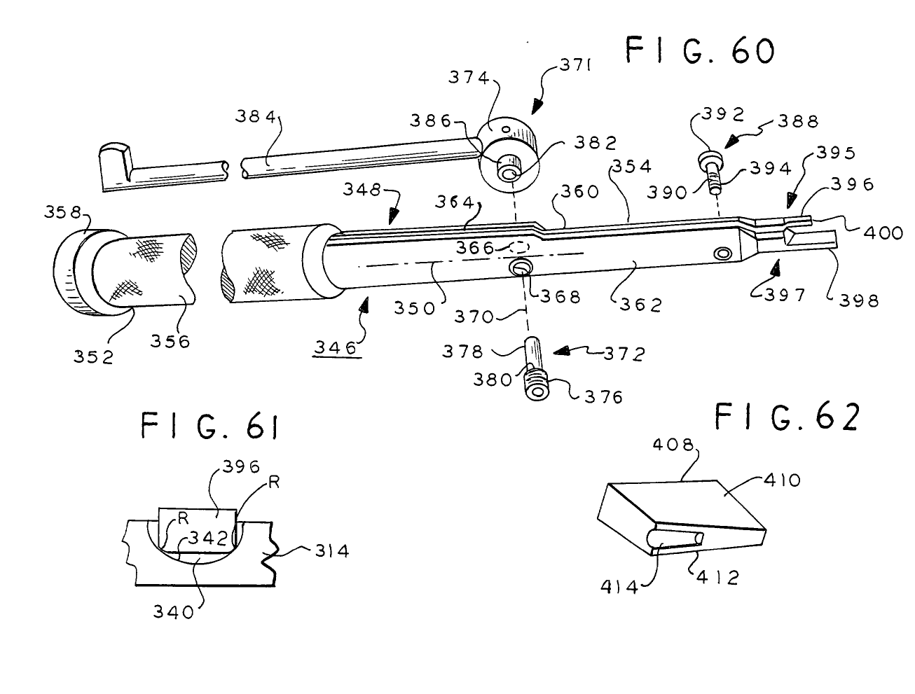

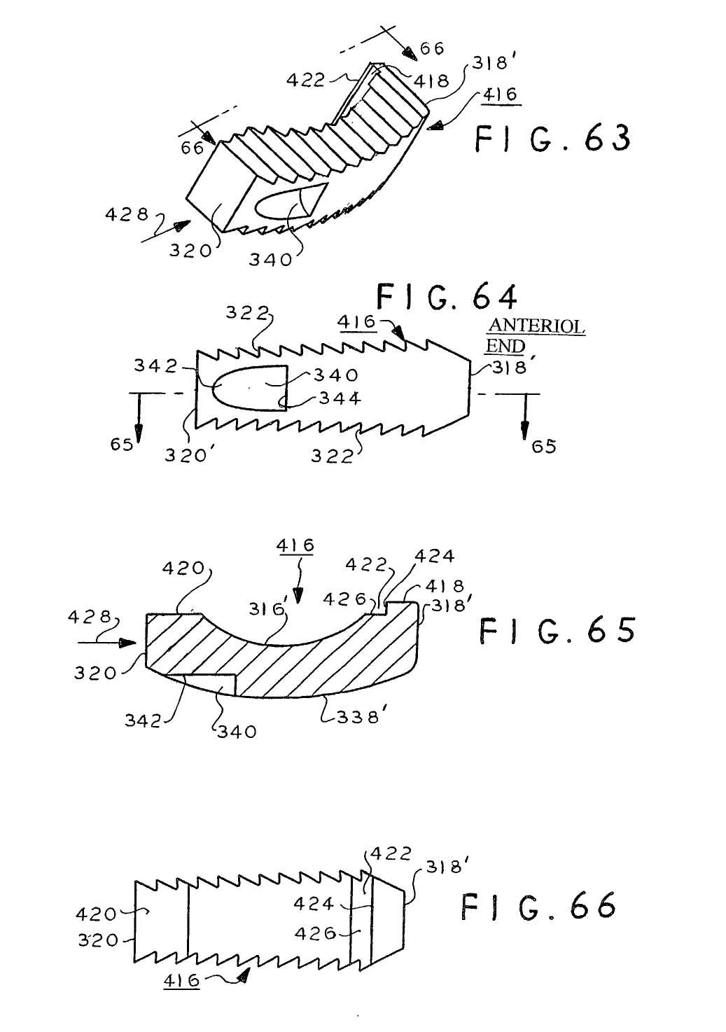

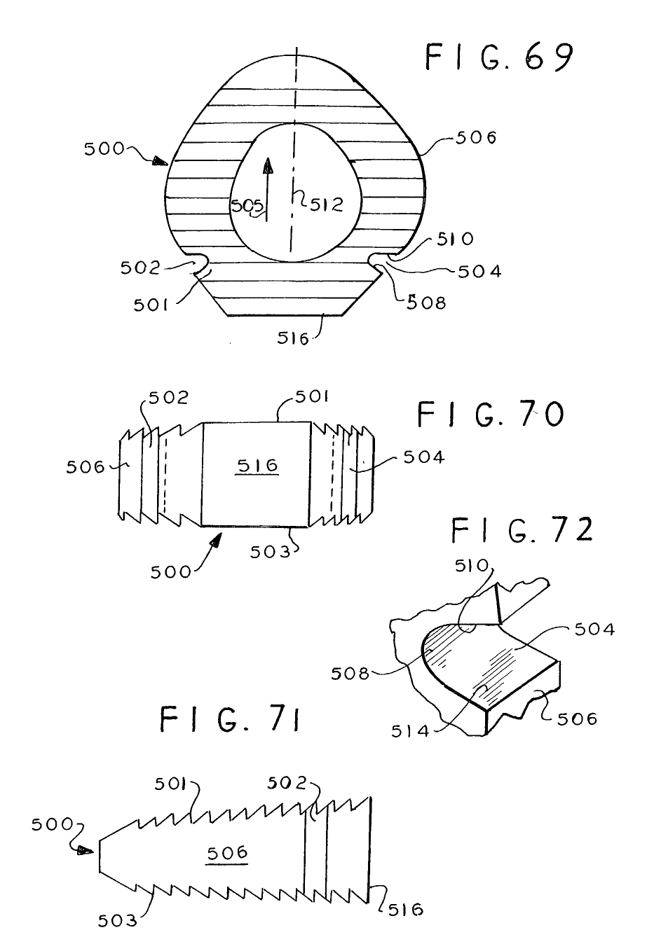

In FIGS. 5-7, in the alternative, implant 50 according to a second embodiment has the same general shape and dimensions as the implant 10 of FIGS. 1-4 except for the recesses 52 and 54. Recess 54 is larger than recess 52 but is of generally the same shape and faces in the same direction 56 opposite the implant insertion direction 58. Recesses 52 and 54 are on opposite sides of the peripheral surface 60. Recesses 54 and 52 have a depth dimension g, FIG. 6. between anterior end surface 61 and semicircular cylindrical wall 62. Wall 62 abuts wall 64 which is planar and is generally parallel to the longitudinal axis 66 of the implant. Wall 62 is normal to axis 66. Wall 62 is defined by a radius R1, which in this embodiment has a value of about 2.5 mm. The walls 64 and 68 are spaced apart distance W2.

Smaller recess 52 has a depth dimension g the same as that of recess 54 between anterior end surface 61 and semicircular cylindrical wall 70. Wall 70 abuts wall 68 which is planar and is parallel to the longitudinal axis 66 of the implant. Wall 70 is normal to axis 66. Wall 70 is defined by a radius, which in this embodiment has a value of about the same as R1. The difference is that the wall 70 has a depth b into the side of the implant peripheral surface 60 that is less than the depth of the wall 62, e.g., about 50%. The asymmetry of the recesses is due to the naturally occurring asymmetry of the bone forming the implant creating different b dimensions in the recesses. However, in all cases the insertion forces are directed through the bone from the anterior side to the posterior side and are not substantially aligned with the medullary canal.

In this case, the mating jaws of the insertion tool have complementary dimensions and shapes to fit in the recesses 52 and 54. The recesses 52 and 54, unlike recesses 24 and 26 of the embodiment of FIG. 1, are not in communication with the top and bottom surfaces 12 and 14, but are recessed between these surfaces.

The walls 62 and 70 receive and abut the tips of the insertion tool whose jaw tips are in contact with these surfaces providing the primary forces needed to insert the implant into the intervertebral space. The walls 64 and 68 are gripped by the mating tool for holding the implant during insertion.

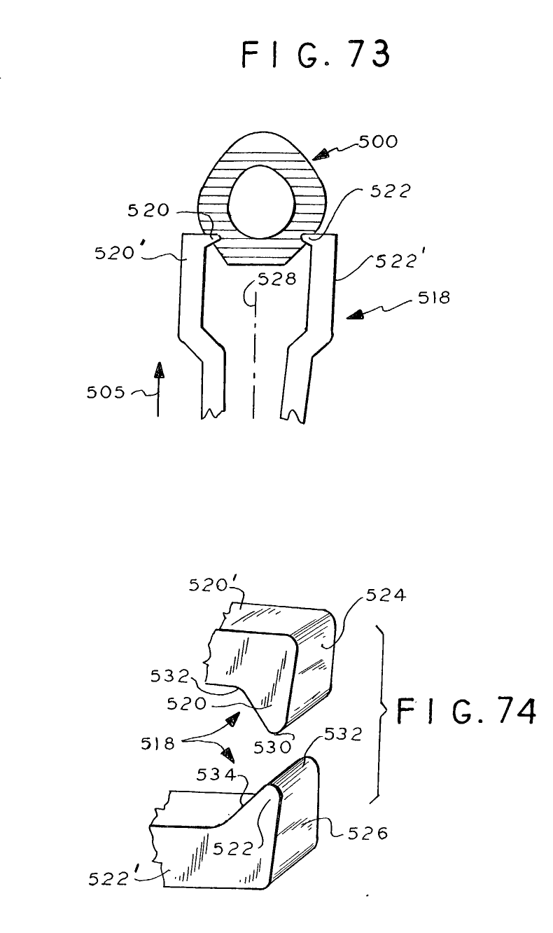

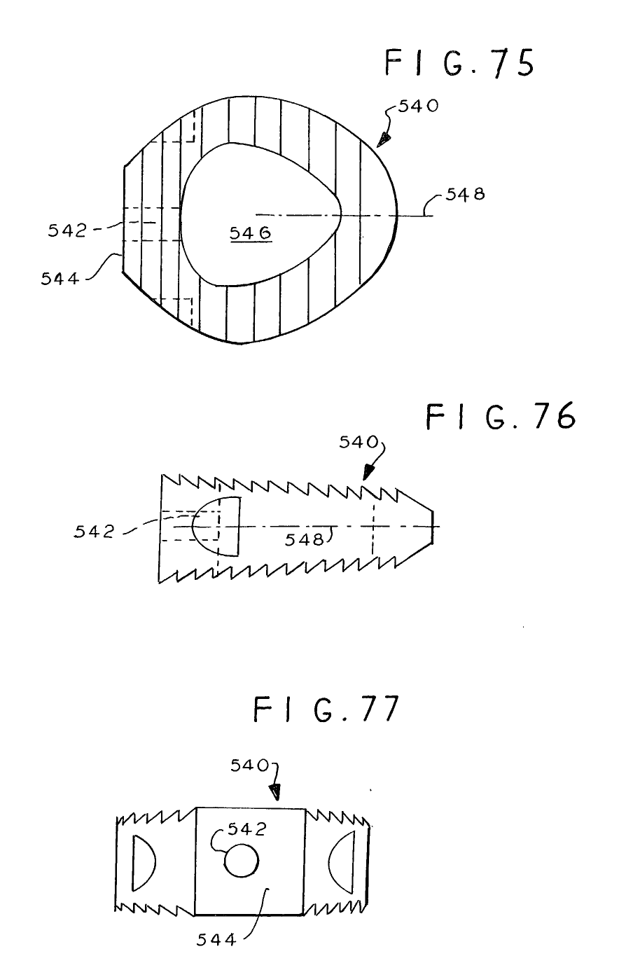

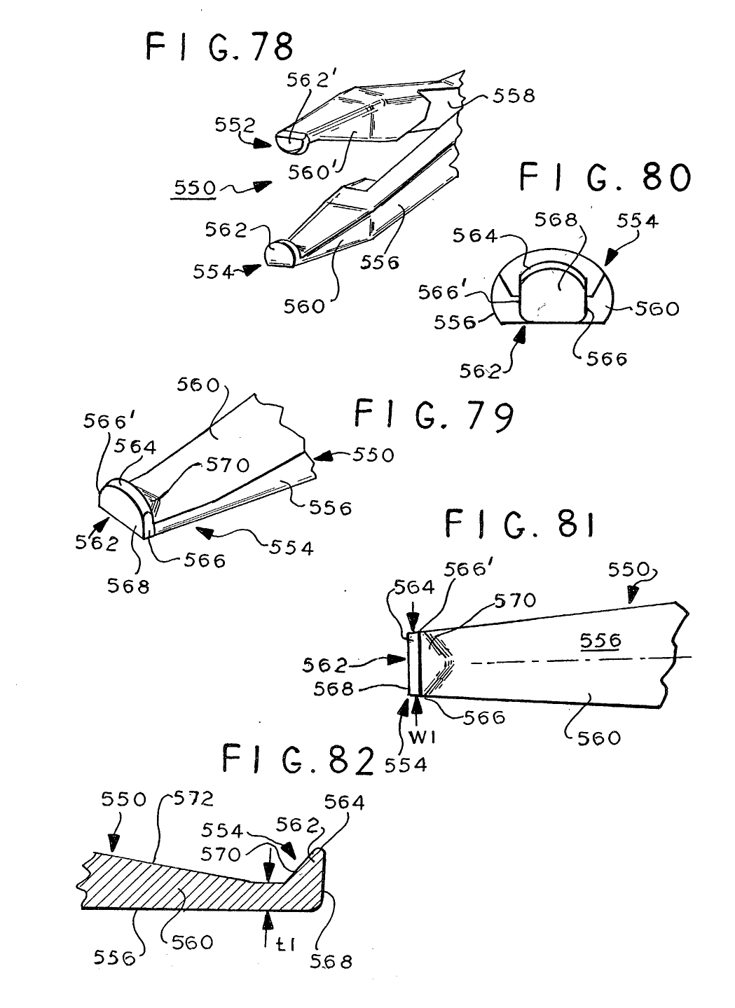

In FIGS. 8-10, in the alternative, implant 72 according to a third embodiment has the same general shape and dimensions as the implant 10 of FIGS. 1-4 and implant 50 of FIGS. 5-7, except for the recesses 74 and 76. Recess 74 is larger than recess 76 but is of generally the same shape and faces in the same direction 82 opposite the implant insertion direction 84. Recesses 74 and 76 are on opposite sides of the peripheral surface 75. Recess 74 has a depth dimension h, FIG. 8, between anterior end surface 80 and wall 86. Wall 86, which is D shaped and the same shape as wall 87 of recess 76, is formed by sides 90 and 92 joined by radii R3. Wall 86 abuts wall 88 which is planar and is parallel to the longitudinal axis 78 of the implant. The two spaced sides 90 and 92 are planar and joined by a central planar wall at the radii R3. The recesses 74 and 76 respective walls 86 and 87 are spaced from end surface 80 distance h. The recess 76 has a planar wall 94 and the recess 74 has a planar wall 88 that are spaced apart distance W3. Recess 74 has a greater depth e than recess 76 depth c into the peripheral surface so that recess 74 is larger than recess 76. Walls 86 and 87 abut and receive the tips of the mating insertion tool for insertion of the implant into the intervertebral disc space. The walls 88 and 94 are gripped by the mating insertion tool to be described for holding the implant during insertion.

In FIGS. 11-13, an implant 98 according to a fourth embodiment is generally of the same material, shape, dimensions and configuration as the implant of FIGS. 1-4 except for the shape, dimensions and configuration of recesses 100 and 102. The recess 100 has a planar wall 104 parallel to recess 102 planar wall 106. Walls 104 and 106 are parallel to axis 109 which is parallel to the insertion direction 1 11. Walls 104 and 106 are spaced apart distance W4 of 25.4 mm (1.0 inches). Recess 102 has a depth q about twice as great as depth q′ of recess 100. Recess 102 has an arcuate wall 108 which is a segment of a circular cylinder and is in communication with top surface 110 and bottom surface 112. Recess 100 has an arcuate wall 114 which is a segment of a circular cylinder and is in communication with top surface 110 and bottom surface 112. Walls 104 and 106 are gripped by and held by the mating insertion tool jaws to be described during insertion and walls 108 and 114 receive and abut the tips of the insertion tool jaws for applying loads during insertion.

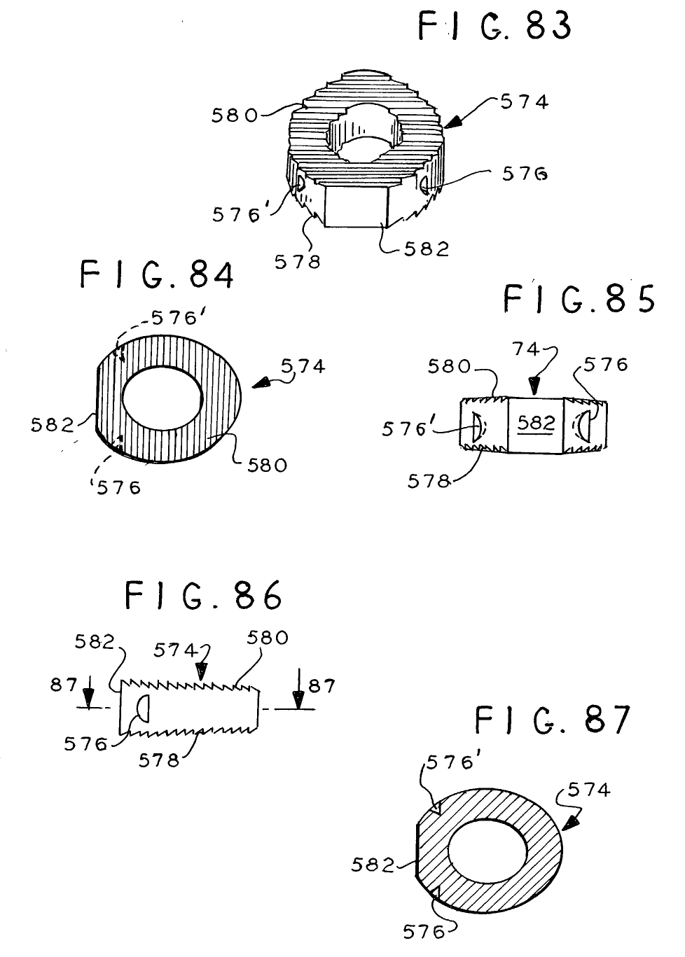

In FIGS. 14-16, an implant 116 according to a fifth embodiment is generally of the same material, shape, dimensions and configuration as the implant of FIGS. 14 except for the shape, dimensions and configuration of recesses 118 and 120 formed in the peripheral surface 122. The recesses 118 and 120 are identical mirror images and the description of recess 118 is representative. Recess 118 has an arcuate wall 124 parallel to recess 120 arcuate wall 126 and facing in opposite directions radially away from the axis 128. Wall 118 has a right semi-cylindrical shape as shown in FIG. 16. Recess 118 is spaced from the top surface 132 and from the bottom surface 134. Recess 118 has a planar wall 130. Recess 120 has a planar wall 136. Walls 124 and 126 are gripped by and held by the mating insertion tool jaws to be described during insertion and walls 130 and 136 receive and abut the tips of the insertion tool jaws for applying loads during insertion.

During surgery, posterior ends of the various embodiments of the implants are inserted first between the adjacent vertebra in the anterior approach. The implants are dimensioned to occupy a substantial portion of the excavated disc space to which the implant is matched. The medullary canal bore may be filled, or partially filled, with any known bone growth promoting material as known in this art.

Such materials may not be bone or may be derived from bone. For example, such materials may include bone chips derived from the patient or not, and/or synthetic materials such as ceramics and metals. However, one such synthetic material such as titanium can fuse to bone. Also, some synthetic materials may also fuse to bone and eventually reform into bone. Examples are calcium phosphates.

Further, there are other synthetic materials that do not fuse to bone, but are replaced by bone. Calcium sulfate and calcium carbonate are examples. Other materials that may be used include polylactic acid (PLA), polyglycolic acid (PGA), polymethylmethacrylate (PMMA), calcium phosphate cement, bioresorbable polymer among others. Thus a wide range of synthetic materials can be used to fill, or partially fill, the implant cavity. The requirements are that they form a mechanical (or chemical) bond to bone, or they can be mechanically fastened to the cortical bone. They are preferably osteoconductive and/or osteoinductive, and either resorb to be replaced by bone, or they contain pores that can be filled with bone. The implants are preferably hard cortical bone which does not generally promote bone growth but provides the desired vertebra support. The implants can be made from surface demineralized cortical bone which will promote bone growth and to provide support to adjacent vertebrae.

The bone chips filling the medullary canal may be formed from the iliac crest from the donor bone or from any other desired source such as chips produced during preparation of the disc site receiving the implant. It is preferred that bone fibers be used. Marrow from other sources may be used to provide cells and active growth factors. Bacteria or DNA techniques may be used to form the bone growth factors in the bone chips or the fibers may be extracted from the marrow or from animal bones.

In FIGS. 17 and 18, insertion tool 140, which is preferably stainless steel, is used for insertion of the implant 116 of FIGS. 14-16. Tool 116 has a pair of elongated arms 142 and 144. Arm 142 has a handle 146 at one end and a jaw 148 at the opposite end. Arm 144 has a handle 150 and a jaw 152 at opposite ends. An extension member 153 extends normal to arm 142 handle 146 at reinforcing gusset 155 which also serves as a stop to limit the motion of the extension portion 159 when the handles are squeezed together. Extension member 153 is made robust, e.g., increased thickness, to receive insertion forces from a hammer (not shown). An extension member 157 extends from the end of handle 150 and has a portion 159 that overlies a portion of the extension member 153. Extension member 157 is also robust of increased thickness as compared to the respective arm 144. The overlying portions of the extension members may abut or be closely spaced to transmit an insertion force to both arms 142 and 144. A hammer blow on the extension member 153 is transmitted to the jaw 148 via pivot mechanism 154 (and also to the jaw 152 via extension member 157).

Pivot mechanism 154 comprises a pivot pin 156 which pivotally joins the arms 142 and 144. The handles, pivot mechanism and jaws of the tool 140 may generally be mirror images of each other except as noted below. Springs 158 and 160 interdigitized at joint 162 and are secured to respective ones of the handles by screws or rivets 163 to urge the handles and arms 142 and 144 apart in a jaw opening and implant releasing direction. The arms have bends 164 and have the shape of conventional pliers. A rod 166 is pivoted to arm 142 and is threaded at end 168 which passes through a passage in arm 144. A nut 170 is threaded to the end 168 to secure the arms in a given desired implant gripping relation. When the arms are displaced toward each other the jaws 148 and 152 are moved together in the direction of the arrows, FIG. 18. Therefore, the nut 170 sets and/or applies the gripping force. The flange on rod 166 limits the nut 170 travel and hence maximum opening distance of the jaws.

In FIG. 19, representative jaw 148, which is identical to jaw 152 and in mirror image relation, includes a jaw extension 172. Extension 172 extends from the arm 142 portion 142′ cantilevered from the pivot mechanism 154 (FIG. 18). The jaw 148 has an implant gripping member 174 which extends from extension 172. The member 174 terminates in tip end surface 176 which is distal the extension 172 and normal to the length dimension of the member 174 in direction 178. Surface 176 abuts and mates with the wall 130 of the implant recess 124 to provide an insertion drive force upon receipt of an insertion force on the arm 142 at extension 153. The member 174 has a generally right semicircular cylindrical surface 180 with parallel saw teeth serration 182 formed by grooves. The jaw can be any shape and may or may not be complementary to the recess gripping surface of the mating implant. The surface 180 does not necessarily mate with surface 124 or 126 (FIG. 14).

For example, in FIG. 19a, the jaw 181 has a generally flattened surface 183 with a radius at each edge 189 that is serrated with serrations 185. In FIG. 19b, the jaw 181 edges 189 tangentially contact the concave arcuate surface of the wall 124. The serrations are optional. Surfaces that transmit insertion forces can be any shape, and not just flat, so long as they adequately transmit these insertion forces.

The members 174,174′ of the two jaws 148,152, FIG. 18, are inserted into the respective implant 116, FIG. 14, recesses 124, 126 for holding and insertion of the implant into the intervertebral disc space in direction 178. Representative surface 180, FIG. 19, may mate with and may be complementary to the recess 124 wall 124 surface, which may be semi-cylindrical or other shapes. Such shapes are not critical. Tangential contact is sufficient for the gripping member to grip the implant. What is important is that the gripping member contacts and grips the implant at the mating gripping surface of the recess regardless of the mating surfaces are complementary or tangential.

The two jaws 148 and 152 cooperate to grip the implant at its recesses. The tip surfaces 176 of the two jaws abut the corresponding walls of the respective recesses such as walls 130 and 136 (FIG. 14). The nut 170 is adjusted to set the gripping forces.

The handlesl46 and 150 are spread apart to release the implant 116 after insertion of the implant.

In FIGS. 21-25, an alternative implant insertion tool 184 is shown for insertion of the implant 116 of FIGS. 14-16 and 20. Insertion tool 184 includes an outer elongated tubular housing 186 which has spaced annular grooves 188 which serve as a gripping handle. Housing 186 at one end has an axially extending cylindrical recess 190, FIG. 23. The housing 186 has an axially extending bore 192 in communication with the recess 190. A larger diameter axially extending bore 194 is in communication with bore 192 at one bore end and with the opposite end 196 of the housing. The bore 194 terminates at housing end 196 in a radially outwardly frusta-conical flared portion 198. The housing flared portion 198 is generally square in its outer periphery as shown in FIG. 21 and is larger in cross section than the remainder of the housing 186.

A rod 200 is located in bore 192 and has a hex head 202 at one end. The rod 200 has threads 204 at its other end. The hex head abuts housing shoulder 206 in the recess 190. Elongated jaw member 208 is located in the bore 194. Member 208 has a threaded bore 210 which is engaged with the threads 204 of rod 200. The member distal the bore 210 is formed with bifurcated branches or arms 212, 214 which can flex with respect to each other in the plane of the drawing sheet, FIG. 23, in directions 216. The arm 212 terminates at jaw 218 and arm 214 terminates at jaw 220. Jaws 218 and 220 may be mirror images and a description of jaw 218 is representative in this case. This due to the fact that the recesses on opposite sides of the implant may differ in shape, location and geometry.

Jaw 220 includes a rectangular in cross section intermediate member 222 extending from arm 214. Jaw implant gripping member 224 extends from the member 222. Gripping member 224′ extends from intermediate member 222′ attached to arm 212. The gripping members are generally parallel to each other. The gripping members 224, 224′ may have the shape and configuration of the gripping member 174, FIG. 19, as discussed according to a given implementation. The gripping members 224, 224′ engage the recesses 124, 126 of the implant 116, FIG. 14. The members have the geometry to roughly mate with and function with the respective recesses of the implants as described. The tips of the gripping members 224, 224′ are used to abut the insertion surfaces of the implant recesses to insert the implant into the disc space.

The intermediate members 222 and 222′ of the respective arms 214 and 212 normally are flexed apart a distance greater than the diameter of the housing flared bore portion 198. The housing portion 198 mates with the members 222 and 222′ in a manner to prevent the jaw member from rotating. The normal position of the intermediate members forces them against the flared bore portion 198 of the housing 186.

A knob 226 has a circular cylindrical drive section 228 which fits in and mates with the housing recess 190, FIG. 23. The drive section 228 has a hex shaped socket 230 which releasably mates with and receives the hex head 202 of the rod 200. The knob receives insertion forces from a hammer to insert the implant if such forces are needed. The insertion forces are transmitted to the opposite end of the tool to the distal tip surfaces of the jaws.

Rotation of the knob 226 relative to the housing either draws the jaw member 208 into the housing 186 or extends the jaw member beyond the housing at end 196. When the jaw member is drawn into the housing bore portion 198, the jaws 218 and 220 are moved together and spread apart when the jaw member is displaced in the opposite direction out of portion 198.

The particular shape of the jaws, FIG. 19, is one arranged to mate with the recesses 124 and 126 of the implant 116. These jaws are reconfigured for the implant insertion tools of FIGS. 17 and 21 according to the shape and configuration of the recesses of the implants of the various embodiments of FIGS. 1, 5 8 and 11. In common with all such implants, the tools of FIGS. 17 and 21 both grip the respective implant at their respective recesses and also provide an insertion force to the implants at the insertion load receiving surfaces of those respective recesses. In all cases, the insertion forces are imposed on the bone implants through the lateral regions of the implant in the direction of the insertion, primarily in the regions between the medullary canal and the implant outer peripheral surface. This minimizes the possible damage to the implant if such forces were exerted more centrally in a direction toward the medullary canal where the implant is the weakest.

In FIG. 27 the directions of the different anterior approaches are shown wherein the lateral approach is normal to the anterior approach and the anterior/lateral approach may vary in the range of about 30-60°, or in general, between the anterior and lateral approach directions, medially between the anterior and lateral approaches as shown by arrow 236.

In FIGS. 28-36, an alternative embodiment of an insertion tool is shown. Tool 238 comprises an outer tubular housing 240, a shaft 242, a jaw section 244 and a thumb screw 246. The shaft 242, FIG. 30 has external threads 248 and a flange 250 at one end. The jaw section 244 has a threaded bore 252 in portion 258 which receives the external threads of the shaft 242. The section 244 is bifurcated into jaws 254, 256 so that the jaws flex relative to the portion 258 of the section 244, directions 260, FIG. 31. The shaft 242 is threaded into the threaded bore 252 which adjusts the length of the shaft and jaw section.

The tubular housing 240 has a longitudinal bore 262 in which the shaft 242 and jaw section are passed through. The housing 240 has an opening 264 which receives the threaded thumb screw 246 which has an internal thread 245. The internal screw thread 245 of screw 246 receives therethrough the threads of the threaded shaft 242.

Rotation of the screw 242 selectively displaces the shaft 242-jaw section 244 combined unit in and out of the bore 262 of the tubular housing 240. The housing 240 has a taper 266 at an enlarged end 268. The taper 266 receives the outwardly flared jaws 254 and 256. As the jaws move in and out of the bore 262 of the housing the taper 266 forces the jaws closed as they move into the housing bore 262 and permit them to spread apart as they move out of the bore 262. The jaws 254 and 256 may have a bend such as bend 230 of the jaws of the tool 184′, FIGS. 24 and 25. Rotation of the thumb screw thus determines and sets the spaced apart distance of the jaws 254 and 256 to grip the implant 270 via its recesses as described above and below herein, such as implant 116 (FIG. 14) and so on.

In FIGS. 3740, implant 272 is a wedge shaped cortical bone ring but may be made of other materials as discussed above. The implant 272 has a flat anterior end surface 274 which identifies to the surgeon that this is the anterior end on anterior/posterior plane 276. Recesses 278 and 280 are aligned on insertion axis 282 which is in the range of approximately 30-60° to the plane 276. See FIG. 27, arrow 236 defining this insertion direction range. The recesses 280, 278 have insertion load receiving surfaces 284, 284′, respectively. Any of the insertion tools described above can be used to insert this implant.

The implant 286 of FIGS. 41-44 is substantially the same as implant 272 of FIG. 37 except that recesses 287 and 288 are of different sizes and locations as shown in FIG. 41. The recesses are different distances 289, 289′ from the end 290. In this case, the tools described above have jaw tips that mate with the insertion surface locations of the implant 272 insertion load receiving surfaces 291, 291′. The anterior end 292 is identified by a flat surface. Further, the gripping surface 293 of recess 287 is arcuate and the gripping surface of recess 288 is flat

In FIGS. 4548, implant 294 has recesses 296, 296′ and a flat surface 298 at the anterior end. This implant is inserted in the lateral direction 300 (see FIG. 27). The recesses have flat insertion load receiving surfaces 302 and arcuate gripping surfaces 304

In FIGS. 49-52, implant 306 is also for insertion in the lateral direction 300. Recesses 308 and 310 differ in size and location as shown. Recess 310 forms the anterior face of the implant in the anterior-posterior direction, arrow 312. The mating insertion tool has jaws that are arranged to apply insertion loads to surfaces 309, 309′ of the respective recesses while gripping the respective gripping surfaces 311, 311′ thereof, the gripping surfaces being generally parallel to the lateral direction of insertion 300 and the insertion load receiving surfaces 309, 309′ being generally parallel to the anterior/posterior direction 312.

In FIGS. 53-56, a C-shaped implant 314 is described in more detail in certain of the applications and patents mentioned in the introductory portion, incorporated by reference herein. The implant 314 is formed from transverse cuts in a long bone such as the femur or other bones as noted in the above-noted patents and applications. The implant 314 is preferably cortical bone but may be other materials, natural or synthetic as also mentioned previously herein above.

The implant 314 is made from approximately one half of a cortical bone ring. The implant 314 has a concave surface 316 formed for example by the medullary canal of the bone. The implant 314 has a flat anterior end surface 318 and a flat posterior end surface 320. The implant 314 has saw teeth 322 on opposing top surface 324 and bottom surface 326 and chamfered surfaces 328 at the anterior end to facilitate insertion in direction 330, FIG. 53.

Implant 314 has two coplanar side surfaces 332, 334, FIG. 55, at the opposite respective posterior and anterior ends of the surface 316. The surfaces 332, 334 extend in the anterior-posterior direction 330 generally parallel to the longitudinal axis 336 of the implant. The implant 314 has a curved convex peripheral surface 338.

Implant 314 has a recess 340 in surface 338 adjacent to and spaced somewhat from the flat posterior surface 320. The recess 340 has a semi-cylindrical insertion tool gripping surface 342 and an insertion tool insertion load receiving surface 344. The surface 344 receives insertion forces in the insertion direction 330 imparted by a tool to be described. This tool grips the surface 342 in a manner to be explained.

In FIGS. 58 and 60, tool 346 is similar to the insertion tool disclosed in the aforementioned copending application serial No. 60/246,601 noted in the introductory portion and incorporated by reference herein. Reference should be made to that application for more details on this tool. In the figures, tool 346

In FIGS. 58 and 60, implant insertion tool 346 comprises an elongated shaft 348 defining longitudinal axis 350 and having a proximal end 352 and a distal end 354. The proximal end 352 comprises a solid metal preferably stainless steel handle 356 having a knurled or roughened gripping surface. The proximal end of the handle is formed into an enlarged disc-like grip member 358. Approximately medially the shaft 348 and extending toward the distal end is a bifurcated portion comprising bifurcated shaft portions 360 and 362 having a gap 364 therebetween.

The shaft portion 360 has a through bore 366. The shaft portion 362 has a threaded bore 368 aligned with bore 366 on axis 370. The threaded bore 368 has a larger diameter than bore 366, which is a smooth surface circular cylindrical bore. A circular recess is formed in a surface of the shaft portion 360 aligned on axis 370 and concentric therewith as are bores 26 and 28.

A displacement member 371 includes a shank portion 372 and a knob 374 connected to a lever 384. Shank portion 372 comprises a threaded stud 376 attached to a smooth walled circular cylindrical shank 378 as a one piece metal element which may also be stainless steel. The stud 376 is larger in diameter than shank 378. Shank 378 is rotatably and slidably mounted in bore 366 and can axially displace in this bore along axis 370. The stud 376 is threaded to bore 368. The threaded stud 376 has a shoulder 380 at the shank 370. This shoulder abuts the shaft portion 360 in the gap 364. The gap 364 may be about 1.5 mm.

Knob 374 is attached to the shank 378 by welding or other fixed securing arrangement after the shank 378 is attached to shaft portion 360 and the stud 376 is engaged in bore 368. The shoulder 380 of the shank portion 372 is located in the gap 364 at the time the shank 378 is attached to the knob 374. The shank 378 is received in bore 382 of the knob 374. The knob 374 and shank portion 372 when fixed then rotate as a unit when the knob 374 is rotated.

The knob 374 is attached to elongated lever 384 to facilitate rotation of the knob. The knob has a right circular cylindrical boss 386 which engages and rotates in a circular cylindrical recess (not shown) in the shank portion 360. The portion 360 is captured between the knob boss 386 and the shoulder 380 of the displacement member 33. The lever 384 helps the surgeon in attaching or releasing the implant 314 thereto.

In operation of the displacement member 371, rotation of the knob 374 axially displaces the stud 376 in the shaft portion 362 along the axis 370. This moves the shoulder 380 against the shank portion 360 along the axis 370. If the member 371 is displaced toward the shaft portion 360, the shoulder 380 will spread the shaft portions 360 and 362 apart widening the gap 364. The shaft portions 360 and 362 bend relative to each other due to flexure of the material at their junction and/or also along the length of the shaft portions.

The location of the flexure depends upon the thickness of the shaft portions. Flexure also may occur at the junction between the two shaft portions. This flexure is resilient so that any bending of the shaft portions results in a bias force tending to return the shaft portions to their quiescent position. This bias force will not cause the shaft portions to return to their quiescent position by itself due to the presence of the displacement member 371. The displacement member via its knob must be rotated to do so. The displacement member 371 actively opens and closes the two shaft portions 360 and 362. In the closing position, the knob 374, when displaced toward portion 362, forces the captured flexed portion 360 to its quiescent position.

Displacement of the displacement member 371 toward shaft portion 362 moves the boss 386 abutting the shaft portion 360 in the mating recess toward the shaft portion 362 and thus displaces the shaft portion 360 also toward shaft portion 362 closing the gap 364.

A stop member 388 comprises a shank 390 and a head 392. The shank 390 is threaded at threaded stud end 394 and is smooth at the head 392 end . The threads of the shank 390 are attached to mating threads in the shaft portion 362 and the head 392 is received in a recess in the shank portion 360. The shank 390 smooth portion is received in a mating bore in the shaft portion 360.

In operation of the stop member 388, the member 388 is threaded into the shank portion 362 a distance so that the head 392 is spaced from the bottom of the recess (not shown) in the portion 360. The shank portions 360 and 362 can spread apart a distance until the head abuts the bottom wall of the recess in the portion 360. This limits the motion of the shaft portions and the amount they can spread apart. This prevents the shaft portions from being spread apart too great a distance which may be undesirable in certain implementations, conditions or uses of the tool 346.

The tool 346 has a pair of jaws 395, 397 respectively formed at the end of the shaft portions 360 and 362. Jaw 395 comprises a one piece integral rectangular in cross section extension 396 extending from shaft portion 360. Jaw 397 comprises a rectangular in cross section extension 398 extending from shaft portion 362. Both extensions extend in the distal direction to the right in FIG. 60. The extension 396 is dimensioned to engage the recess 340 in the implant 314, FIG. 57. The extension 398 is dimensioned to abut the flat surfaces 332 and 334 at flat gripping surface 399 (FIG. 59) gripping the implant at these surfaces and bridges the concave surface 316 of the implant 314, FIG. 57.

The extension 396 has a flat rectangular tip 400 abutting the implant insertion load receiving surface 344 of the recess 340 (FIGS. 53-55), and grips the implant at the implant recess gripping surface 342. The extensions may be relatively thin elements, e.g., about 2 mm thick. In FIG. 61, the extension 396 may be generally rectangular with radii R at its corners abutting the recess surface 342 of the implant 314. In the alternative, the extension 396 may be complementary to the recess surface 342 contour. In a further alternative, the extension may have a triangular or curved surface which tangentially contacts the recess surface 342 in a line contact. In all cases, the tip 400 is blunt for imparting an insertion force on the recess insertion load receiving surface 344.

The jaws 395 and 397 each have a shoulder 406 and 408, respectively, extending normal to the extensions 396 and 398. The shoulder 404 may abut the implant 314 posterior surface 320, FIG. 57. The shoulder 406 is spaced from the implant surface 320. The shoulder 404 forms a further flat insertion load receiving surface for insertion of the implant into the disc space. The use of the shoulder 404 as an insertion wall is optional.

In FIG. 62 a further implant 408 is formed of bone or other material and is rectangular in transverse section. The implant 408 has tapered top and bottom surfaces 410 and 412 forming it into a wedge. A recess 414 of the type described above for the different implants is in one or both opposite sides of the implant. The recess may have a curved or flat gripping surface and has a flat insertion load receiving surface for receiving the tip of the insertion tool jaw gripping member.

FIGS. 63-66 illustrate a further implant 416 also of C-shape as the implant 314 of FIGS. 53-56. The implant 416 however has flat parallel side surfaces 418 and 420 which lie in different planes. Reference numerals that are the same refer to the same parts in the implants 416 and 314, whereas reference numerals with primes but otherwise the same refer to similar parts. The surface 418 has a recess 422 with normal insertion wall surface 424 and gripping surface 426. The insertion direction 428 is in the posterior-anterior direction.

In FIG. 67, implant 416 is inserted by insertion tool 346′ via jaws 396 and 398′. Jaw 398′ fits into the recess 422 for gripping the surface 426 and insertion load receiving surface 424, FIG. 65. Jaw 396 engages the recess 340 which may be the same as recess 340 in implant 416, FIGS. 63-66. Both jaws 396 and 398′ apply insertion loads to the implant on opposite sides. The shoulder 430 of jaw 398′ may also insert the implant at end surface 320′.

In FIG. 69, anterior approach implant 500 according to a further embodiment has the same general shape and dimensions as the implant 10 of FIGS. 1-4 except for the recesses 502 and 504. Recesses 502 and 504 are of generally the same shape and dimensions and not necessarily mirror images of each other because of the uneven geometry of the bone geometry. Recesses 502 and 504 form channels that extend generally normal to the respective top and bottom surfaces 501, 503 and are on opposite sides of the peripheral surface 506.

Representative recess 504 has an inner portion 508 which is generally semicircular in cross section. Wall portion 508 is continuous with planar impact wall 510 which is on the posterior side of the recess 504 and is generally normal to the longitudinal anterior-posterior direction axis 512 of the implant. Wall 508 has a further side wall 514 which extends continuous with the bottom wall portion 508. Wall 514 extends on the anterior side of the recess 504 toward the outer peripheral surface 506 inclined at an acute angle to the axis 512 and toward the anterior flat surface 516. The recesses 502 and 504 are linear as shown in FIG. 70 and approximately equally spaced from axis 512 depending upon the symmetry of the implant about axis 512. The recesses also are in communication with the implant 500 top and bottom surfaces 501 and 503. Again, like above, the placement of the dovetail cut-outs will depend on the natural bone geometry. The dovetails will be placed to minimize the impact force through the interior portion of the implant over the medullary canal and, therefore, will not necessarily be mirror images.

The wall 510 receives insertion impact loads applied by the insertion tool 518, FIGS. 73, in direction 505 a manner similar to that described above for the implant 10 of FIG. 1, implant 50 of FIG. 5 and so on. Those implants are inserted by the tools of FIGS. 17 and 28, for example.

The mating jaws 520′, 522′, FIGS. 73 and 74, of the insertion tool 518 have complementary dimensions and shapes to fit in the respective recesses 502 and 504. The wall portion 508 and wall 510 receive and abut the tips 520, 522, FIG. 73, of the insertion tool 518 jaws 520′ and 522′, respectively. These jaw tips have surfaces in contact with the surfaces of these recess walls providing the primary forces needed to insert the implant into the intervertebral space. The wall portion 508 receives the gripping forces of tips 520 and 522 in a direction normal to the axis 512. These gripping forces grip the implant during insertion. The tips 520 and 522 have end surfaces 524 and 526 normal to the longitudinal axis 528 of the insertion tool 518. The end surfaces 524 and 526 are used to impact the impact receiving surfaces of walls 510 of the implant, FIGS. 72, 74.

In FIG. 74, the tips have semi-cylindrical surfaces 530 and 532 which preferably mate with the recess bottom wall portion 508. The tips may have surfaces 530 and 532 of radii smaller than the radii of the portion 508. While the surface of portion 508 may be a portion of a circle, it may have any curvature as desired. The tips 520 and 522 have a rear surface 532, 534 respectively which may abut the surface 514, FIG. 72, of the recesses 502 and 504, respectively.

The jaws 520′ and 522′ may be used with an insertion tool such as tool 140, FIG. 17, or tool 238, FIG. 28 or a tool of any other suitable configuration for impact insertion of the implant.

In FIGS. 75-77, implant 540 is the same as implant 50 of FIG. 5, and in the alternative, may be constructed as any of the other implants of FIGS. 1, 8, 11, 14 and so on. Implant 540 has a through bore 542 between the anterior planar surface 544 and central opening 546 on longitudinal axis 548. The bore 542 may be threaded or unthreaded. The bore 542 receives the stud 438 of the tool 430, FIG. 68. The stud is preferably not threaded for use with an unthreaded bore 542.

In this way, the stud and bore serve to stabilize the orientation of the implant to the insertion tool while permitting the insertion tool jaws to apply the entire insertion impact forces against the implant in the mating implant insertion tool jaw receiving recesses. When the bore 542 and stud are not threaded, there may be some clearance between the two so that all of the impact forces are transmitted to the jaws and implant impact receiving recesses. The bore 542 may be about 3.18 mm (0.125 inches) in diameter. The implant of FIGS. 69 and 75, as also the other implants described above of these configurations, may have an anterior end height in the range of about 11-19 mm and a length, and in FIGS. 70 and 76, from left to right in the figures, in ranges of about 24-27 mm and 28-30 mm. The implants may have a transverse width normal to the longitudinal axes thereof of about 24-28 mm. The top and bottom surfaces converge at an angle of about 8°. The posterior insertion end of the implants is chamfered as shown.

FIGS. 78-82 illustrate a spinal implant insertion tool 550 according to a further embodiment of the present invention. The tool 550 may have the overall actuating mechanism configuration of tool 140, FIGS. 17-19, but has implant gripping jaws 552 and 554 with a different configuration as shown in FIGS. 78-82. In the alternative, still other actuating mechanisms such as shown herein or as known in the prior art may be used with the implant insertion jaws 552 and 554 of FIGS. 78-82.

The tool 550 implant gripping jaws 552 and 554 are preferably identical in this implementation and in mirror image relation, but may differ from each other in other implementations. Representative jaw 554 extends from arm 556 of tool 550. Jaw 552 extends from arm 558. The arms 556 and 558, by way of example, are pivotally connected (not shown) as shown for tool 140, FIG. 17, and have the same or similar handle construction (not shown) as tool 140. Jaw 554 is representative of jaw 552 in mirror image relation and therefore jaw 552 will not be described separately.

Jaw 554 extends from arm 556 via a tapering jaw section 560 which converges in cross section area toward the implant gripping member 562. Jaw 552 has a gripping member 562′ which is identical to but in mirror image relation to gripping member 562. Section 560 is rectangular in cross section and has a relatively thin thickness t1 and a broad width w1. The gripping member 562 has an arcuate peripheral implant gripping surface 564. The surface 564 terminates at flat opposite side walls 566, 566′. The surface 564, in side elevation as shown in FIG. 82, is also arcuate and formed by a radius between the element 562 end wall 568 and the opposite inclined wall 670 facing and terminating at the arm section 560. Wall 570 is triangular in plan view, FIG. 81, and comprises two surfaces inclined at angles relative to each other as they extend to and between walls 566, 566′. The wall 570 is inclined from the element tip surface 564 to its base region at surface 572 of the arm section 560, FIG. 82. The implant gripping member 562 thus comprises a number of complex curved surfaces projecting upward from the arm surface 572 and extending between the side walls 566, 566′ and end wall 568.

Implant 574, Figs,. 83-87, has recesses 576, 576′ which are identical but in mirror image relation. The recesses 576, 576′ mate with and receive the insertion tool 550 respective corresponding jaw gripping members 562, 562′, FIGS. 78-82. The implant 574, except for the recesses 576, 576′, may be identical to the implant 500, FIG. 69, for example, or identical to any other of the disclosed implants herein except for the recesses 576, 576′. The recesses 576, 576′ are complementary to the respective gripping members 562, 562′ of the insertion tool 550 and generally have the cross section shape of recesses 502, 504 of implant 500, FIG. 69. The difference between recesses 502, 504 and recesses 576, 576′ is that the recesses 576, 576′ are spaced from the bottom surface 578 and top surface 580 of the implant 574. The recesses 576, 576′ face and are near the anterior end 582 of the implant, which end is a flat surface.

In operation, the implant gripping members 562, 562′ of jaws 552 and 554, FIG. 78, are inserted into the recesses 576, 576′ of the implant 574, FIG. 83. The recesses 576, 576′ are formed as dimple depressions in the outer peripheral side wall of the implant 574. The gripping members 562, 562′ are inserted into the respective corresponding depressions and are surrounded on all sides by the side wall of the mating recess 576, 576′.

The end wall surface 568 of the gripping members serves as an insertion impact surface as described above for impacting against the mating recess 576, 576′ surface to insert the implant into the disc space. However, the member 562 is surrounded by the recess 576 walls which enables the member 562 to firmly grip the implant in all directions. For example, in the implant 500 of FIG. 69, its recesses 502 and 504 are in communication with the bottom and top surfaces of the implant and has a surface parallel to the anterior/posterior axis. Thus the implant could slide off of the jaws in the anterior direction. The same is true of the implant 10 of FIG. 1. In the case of the implant 574 of FIG. 83, however, the implant can not slip off of the jaw elements 562 in any direction. This permits the surgeon to manipulate the insertion tool 550, FIG. 78, and gripped implant 574 in any direction even after the implant is inserted into the disc space without the implant moving relative to the jaw members 562. This is important in the situation where the implant needs to be maneuvered somewhat after it is inserted into the disc space and is compressed by the adjacent vertebrae after the distraction of the vertebrae is removed. The vertebrae provide increased resistance to such implant maneuvering which might cause jaws of certain of the tools disclosed herein to be less able to apply sufficient force to move the implant in certain directions. This is especially true wherein the recesses have surfaces parallel to the anterior/posterior axis such as implant 10, FIG. 1, for example. This will not occur with the tool 550 of FIG. 78 and implant 574 of FIG. 83. The recess configuration of implant 550, FIG. 78, may be used in any of the embodiments of the implants disclosed herein.

It will occur to one of ordinary skill that modifications may be made to the disclosed embodiments without departing from the scope of the invention as defined in the appended claims. The disclosed embodiments are given by way of illustration and not limitation. For example, while circular semi-cylindrical jaws are disclosed for mating with complementary shaped recesses, other convex shapes may also be used such as oval and other non-circular surfaces as well as planar surfaces for use with the implants of FIGS. 1, 5, 8 and 11.

The surfaces of all such jaws engaging the implant may be knurled or have serrations as described or other roughened surfaces to better grip the implant. These surfaces may also be smooth as desired. Still other insertion tools of different configurations than those shown may be used with the recesses of the different disclosed implants, the important aspect comprising the shape and configuration of the jaws rather than the particular mechanism for operating the jaws. In all cases, the jaws of the insertion tool have tips arranged to apply insertion loads against the mating insertion load receiving surface of the corresponding implant recess. In some cases, the jaws may have flat implant gripping surfaces for gripping flat recess surfaces extending in the anterior-posterior direction. But, in all cases, the recesses each have an insertion load receiving surface that is generally transverse to the anterior-posterior direction for mating with the jaw tips.

In a further embodiment, the insertion tool may have a threaded rod that mates with a threaded bore in the implant for holding the implant during insertion. Such a rod is shown for example in U.S. Pat. No. 5,522,899 to Michelson incorporated by reference herein. In this case, the insertion tool jaws have tips that engage the insertion load receiving surfaces of corresponding recesses in the implant plug. The jaws however do not grip the implant plug which gripping function is carried out by the threaded rod. Such a threaded rod is also disclosed in copending application serial No. 60/246,297 mentioned in the introductory portion and also incorporated by reference herein.

An example of such a tool is also shown in FIG. 68 wherein insertion tool 430 is similar to tool 140 of FIG. 17 except as modified below, the parts with the same numbers being the same. The handle 432 has an extension 434 which receives and supports a rod 436, which may be rotatable or move just in translation. Rod 436 has a threaded stud 438. The jaws 440 and 442 are use to insert a received implant plug 444 at mating recesses 446 (one being shown). However, the jaws 440 and 442 do not grip the plug 444. The rod 436 rotatably passes through a mating bore (not shown) in the pivot member 448 and axially extending channels (not shown) in the arms 450 and 452 connected to the handles 432 and 432′. The channels permit the arms to pivot about pivot member 448. The rod 436 stud 438 engages a threaded bore 454 in the plug 444, holding the plug during insertion. It should be understood that the implants that are inserted laterally or anterior/laterally may be inserted on either side of the patient, the particular orientation shown in FIG. 27 being by way of example.

It should be understood that the term “anterior/posterior” axis and positions are used herein for purposes of reference and are not limited to spinal bone posterior and anterior positions. These terms are used herein and in the claims as terms of reference for relating the various locations of an implant to the recipient bone site. While the implants made of bone are described as having weak sections that are aligned with a central chamber formed in the implant and stronger sections or regions that are aligned with the implant material adjacent to the chamber, it should be understood that synthetic implants may also have relatively weak and strong regions therein. The insertion recesses provided such implants are provided in the stronger regions to preclude insertion damage to the implants that might otherwise occur in the presence of impact or other insertion forces.

In addition, while the implants described herein are preferably for use in spinal applications, it will occur to those of ordinary skill that the principles of the present invention may be applied to implants into bone that are not spine related. Such applications include implants where impact or insertion loads are required creating sufficient insertion forces that could damage the implant during insertion. 525400-284

What is claimed is:

1. An implant for fusing and/or supporting bone of a human or animal defining an implant receiving space and defining anterior and posterior positions with respect to the recipient implant receiving space, the implant for insertion into the implant receiving space in an insertion direction, the implant comprising:
a body having opposing top and bottom surfaces and a peripheral outer surface intermediate the top and bottom surfaces, the top and bottom surfaces for engaging bone of said implant receiving space, the body having an anterior end and a posterior end defining an anterior/posterior axis corresponding to the implant receiving space respective anterior and posterior positions;
the peripheral outer surface having at least one recess having a first surface for receiving a body gripping force transverse to the implant insertion direction and a second insertion load receiving surface transverse to the first surface and transverse to the implant insertion direction for insertion of the body into the implant receiving space in the insertion direction.

2. The implant of claim 1 wherein the peripheral outer surface has a planar surface at and defining the anterior end, the at least one recess being spaced from the planar surface.

3. The implant of claim 1 wherein the at least one recess is located on the body for insertion of the body in a direction transverse to the anterior/posterior axis of the vertebral bone.

4. The implant of claim 3 wherein the at least one recess is located on the body for gripping and insertion in an insertion direction in the range of about 0° to about 90° to the anterior/posterior axis.

5. The implant of claim 1 wherein the body has regions of differing strengths in an insertion direction through the outer peripheral surface such that an insertion load at a relatively weak region may damage the implant, the at least one recess being located at a region of greater strength than the weak region.

6. The implant of claim 1 wherein the body has a generally central chamber in communication with the top and bottom surfaces, the at least one recess being axially aligned on an axis passing through the body on a body side wall between the chamber and the outer peripheral surface.

7. The implant of claim 1 wherein the body is bone.

8. The implant of claim 6 including a pair of said recesses aligned on a corresponding axis passing through the body at opposite sides of the chamber.

9. The implant of claim 1 wherein the body is C-shaped and having a first peripheral side wall surface between the top and bottom surfaces extending between the anterior and posterior ends and a second peripheral side wall surface opposite the first side wall surface extending between said ends and between said top and bottom surfaces, the second peripheral side wall surface being defined by first and second planar surfaces interrupted by an intermediate concave surface, the at least one recess being located in the first peripheral side wall surface.

10. The implant of claim 9 wherein the at least one recess is located adjacent to the posterior end.

11. The implant of claim 10 wherein the first planar surface is adjacent to the anterior end of the body and the second planar surface is adjacent to the posterior end of the body, the body including a further of said at least one recess in the first planar surface,.

12. The implant of claim 1 wherein the body gripping first surface is arcuate.

13. The implant of claim 1 wherein the body gripping first surface is a semi-cylindrical channel.

14. The implant of claim 1 wherein the gripping first surface is planar and the second surface is planar transverse to the first surface.

15. The implant of claim 1 including a plurality of identical said at least one recess.

16. The implant of claim 1 including a plurality of said at least one recess wherein the plurality of recesses are of generally the same shape, but have different dimensions.

17. The implant of claim 1 including a plurality of said at least one recess wherein the plurality of recesses have differently shaped gripping surfaces.

18. The implant of claim 1 including a plurality of said at least one recess, the recesses each having a different relative location to each other in the insertion direction.

19. The implant of claim 1 wherein the at least one recess is adjacent to the anterior end of the body and including a further of said at least one recess adjacent to the posterior end of the body.

20. The implant of claim 1 wherein the at least one recess is in communication with the top and bottom surfaces.

21. The implant of claim 1 wherein the at least one recess is spaced from the top and bottom surfaces.

22. The implant of claim 1 wherein the peripheral surface except for the recess is generally curved.

23. The implant of claim 1 wherein the body has a planar anterior surface and a curved peripheral outer surface extending between the top and bottom walls interrupted by said planar anterior surface and by said at least one recess.

24. The device of claim 1 wherein the at least one recess is D-shaped in side elevation view.

25. The implant of claim 1 wherein the at least one recess has a third surface that terminates at the first surface, the third surface being arcuate.

26. An insertion tool for holding and inserting an implant in an insertion direction for fusing and/or supporting vertebral bone comprising:
first and second jaws movable in an implant gripping and release directions respectively toward and away from each other, each jaw having a first gripping surface for gripping an implant second surface, each jaw having a tip surface at the terminal end of the first jaw distal the mechanism means set forth below, the tip surface for engaging the implant second surface for imparting an insertion load on the implant to insert the implant for said fusing or supporting; and
mechanism means for manually moving said jaws in said directions.

27. The tool of claim 26 wherein the mechanism means comprises first and second arms movably secured relative to each other and terminating at first ends distal the jaws, resilient means for resiliently biasing the arms apart in a implant release position and holding means for holding the arms against the bias of the resilient means in a implant gripping position, the first implant gripping surfaces for cooperatively gripping the implant and holding the implant during insertion of the implant.

28. The tool of claim 27 wherein each said arms includes a first arm portion extending transverse to that arm at a region distal the first and second jaws, the arm first portions having at least a further portion, the further portions overlapping.

29. The tool of claim 28 wherein at least one of the arm first portions is arranged for receiving an insertion load force for driving the implant into a spinal disc space.

30. The tool of claim 26 wherein one of said first and second jaws has a planar implant gripping surface and the other of the first and second jaws has a non-planar implant gripping surface.

31. The tool of claim 30 wherein the non-planar implant gripping surface of the other jaw is arranged to tangentially abut the implant first surface.

32. The tool of claim 31 wherein the non-planar surface is curved.

33. The tool of claim 27 wherein each of the arms has a longitudinal axis, each arm first portions being transverse to that arm longitudinal axis, the one arm first portion being joined to its arm by a stop for limiting relative displacement of the other arm first portion.

34. The tool of claim 26 wherein the mechanism means comprises:
a tubular housing;
a jaw member in the housing having a threaded bore at a first end and first and second arms extending toward a second end opposite the first end, the arms extending beyond the housing and each terminating in a respective jaw, each arm being resilient relative to the first end;
a rod in the housing threaded to the threaded bore at a first rod end and terminating in a projection at a second rod end distal the rod first end, the housing having a recess adjacent to the projection; and
a knob for mating with the recess and for mating with the projection for rotating the rod relative to the jaw member to thereby displace the jaw member relative to the housing axially along the housing, the housing and arms being arranged to selectively open and close the jaws in response to said relative axial displacement of the jaw member to the housing.

35. The tool of claim 26 wherein the tool includes first and second branches movably secured relative to each other, each branch defining a longitudinal axis, ach branch terminating in a jaw, each jaw including first and second portions extending from the corresponding branch, the first portion extending in a first direction along the branch longitudinal axis, the second portion being oriented at an acute angle relative to the first direction.

36. An insertion tool for holding and inserting an implant in an insertion direction for fusing and/or supporting bone comprising:
first and second jaws movable in directions respectively toward and away from each other, each jaw having a tip surface at the terminal end of the first and second jaws distal the mechanism means set forth below, the tip surface for engaging an implant first insertion load receiving surface for insertion of the implant relative to said bone for said fusing and/or supporting;
mechanism means for manually moving said jaws in said directions; and a rod secured to the mechanism means for releasably holding the implant in a position for engagement by said jaws.

37. The tool of claim 36 wherein the rod has a threaded stud for engagement with a threaded bore in the implant.

38. The tool of claim 36 wherein the mechanism means includes first and second handles pivotally secured together and an extension member secured to one of the handles, the rod being rotatably supported by the one handle, the first jaw being connected to the first handle and the second jaw being connected to a second handle.

39. The tool of claim 36 wherein the tool includes first and second branches movably secured relative to each other, each branch defining a longitudinal axis, each branch terminating in a different one of said jaws, each jaw including first and second portions each extending from the corresponding branch extending in a direction along the branch longitudinal axis.

40. The implant of claim 1 wherein the first surface is arcuate and further including a third surface facing the second surface, the third surface being inclined relative to the second surface and extending away from the second surface in a diverging relationship from said first surface.

41. The implant of claim 1 wherein the at least one recess is concave with a bottom wall portion and two side wall surfaces extending from the bottom wall portion forming a channel, the second surface forming one of said two side wall surfaces, a third surface facing the second surface and forming the other of said two side wall surfaces.

42. The implant of claim 1 wherein the at least one recess is a channel with a bottom wall portion and two facing spaced side walls extending from the bottom wall portion.

43. The tool of claim 26 wherein the jaws have tips shaped to be received in a 10 channel having a bottom wall and two spaced side walls extending from the bottom wall.

44. An implant for fusing and/or supporting bone of a human or animal defining an implant receiving space and defining anterior and posterior positions with respect to the recipient implant site, the implant comprising:
a body having a peripheral outer surface formed by at least one peripheral side wall and opposing top and bottom surfaces, the top and bottom surfaces for engaging adjacent bone of said implant receiving space, the body having an anterior end and a posterior end defining an anterior/posterior axis corresponding to the recipient implant site respective anterior and posterior positions, the axis defining a plane between said top and bottom surfaces that is approximately equidistant from the top and bottom surfaces;
the body exhibiting different degrees of strength in corresponding different peripheral regions in respect of an insertion force applied to the body in said plane in an insertion direction for inserting the body into said implant receiving space, at least one of said different peripheral regions being the weakest in respect of said insertion force;
the at least one side wall having at least one recess located at a peripheral region exhibiting a strength in said plane in said insertion direction greater than said at least one weakest region for receiving said insertion force to thereby minimize damage to the body during said insertion.

45. The implant of claim 44 wherein the insertion force defines an implant insertion axis, the body having a gripping first surface for receiving a body insertion gripping force applied to the body in a direction generally normal to the insertion axis.

46. The implant of claim 44 wherein the body is bone.

47. The implant of claim 44 wherein the body is cortical bone.

48. The implant of claim 44 wherein the implant is formed by a transverse slice of the diaphysis of a long bone.

49. The implant of claim 44 wherein the implant is arranged for fusing vertebrae.

50. The implant of claim 44 including a plurality of said at least one recess.

51. The implant of claim 44 wherein the at least one recess has a first surface for receiving a gripping force and a second surface transverse to the first surface for receiving an implant insertion force.

52. The implant of claim 44 wherein the at least one recess is in the at least one side wall intermediate the anterior and posterior ends.

53. The implant of claim 52 including a second recess in a further side wall opposite the at least one side wall.

54. The implant of claim 53 wherein the at least one recess and second recess are mirror images and the same shape.

55. The implant of claim 53 wherein the at least one recess and second recess are mirror images and different in shape.

56. The implant of claim 44 wherein the body has chamber, the chamber being aligned with a first region of the body in an insertion direction and transversely adjacent to a second region of the body with respect to the insertion direction, the at least one recess being located to provide an insertion load in the second region spaced from the chamber.
